(19) United States
(12) Patent Application Publication
(10) Pub. No.: US 2014/0364784 A1
(43) Pub. Date: Dec. 11, 2014

(54) TIME-BASED CONTROL OF ACTIVE TOSO SUPPORT

(71) Applicant: Elwha LLC, Bellevue, WA (US)

(72) Inventors: Roderick A. Hyde, Redmond, WA (US); Jordin T. Kare, Seattle, WA (US); Dennis J. Rivet, Chesapeake, VA (US); Lowell L. Wood, JR., Bellevue, WA (US)

(21) Appl. No.: 13/910,511

(22) Filed: Jun. 5, 2013

(51) Int. Cl. A61F 5/02

(52) U.S. Cl. CPC A61F 5/02
USPC 602/19

(57) ABSTRACT

An active torso support is described which is capable of applying force to a torso of a subject with controlled delay and/or duration, in response to user input, sensed events, or signals from a timing device, for example. Sensed events can include, but are not limited to changes in gait, posture, or activity of the subject. Related methods and systems are described.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application is related to and/or claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)). In addition, the present application is related to the “Related Applications,” if any, listed below.

### PRIORITY APPLICATIONS

None.

### RELATED APPLICATIONS

U.S. patent application Ser. No. 13/721,474, entitled POSTURE DEPENDENT ACTIVE TORSO SUPPORT, naming RODERICK A. HYDE, JORDIN T. KARE, DENNIS J. RIVET, AND LOWELL L. WOOD, JR. as inventors, filed 20 Dec. 2012 with attorney docket no. 1108-004-001-000000, is related to the present application.
U.S. patent application Ser. No. 13/739,868, entitled POSITION SENSING ACTIVE TORSO SUPPORT, naming RODERICK A. HYDE, JORDIN T. KARE, DENNIS J. RIVET, AND LOWELL L. WOOD, JR. as inventors, filed 11 Jan. 2013 with attorney docket no. 1108-004-012-000000, is related to the present application.
U.S. patent application Ser. No. 13/748,871, entitled GAIT-RESPONSIVE ACTIVE TORSO SUPPORT, naming RODERICK A. HYDE, JORDIN T. KARE, DENNIS J. RIVET, AND LOWELL L. WOOD, JR. as inventors, filed 24 Jan. 2013 with attorney docket no. 1108-004-009-000000, is related to the present application.
U.S. patent application Ser. No. 13/875,538, entitled EXTERNAL SENSOR-BASED CONTROL OF ACTIVE TORSO SUPPORT, naming RODERICK A. HYDE, JORDIN T. KARE, DENNIS J. RIVET, AND LOWELL L. WOOD, JR. as inventors, filed 2 May 2013 with attorney docket no. 1108-004-010-000000, is related to the present application.

The United States Patent Office (USPTO) has published a notice to the effect that the USPTO's computer programs require that patent applicants reference both a serial number and indicate whether an application is a continuation, continuation-in-part, or divisional of a parent application. Stephen G. Kunin, Benefit of Prior-Filed Application, USPTO Official Gazette Mar. 18, 2003. The USPTO further has provided forms for the Application Data Sheet which allow automatic loading of bibliographic data but which require identification of each application as a continuation, continuation-in-part, or divisional of a parent application. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant has provided designation(s) of a relationship between the present application and its parent application(s) as set forth above and in any ADS filed in this application, but expressly points out that such designation(s) are not to be construed in any way as any type of commentary and/or admission as to whether or not the present application contains any new matter in addition to the matter of its parent application(s).

To the extent that the listings of applications provided above may be inconsistent with the listings provided via an ADS, it is the intent of the Application to claim priority to all applications listed in the Priority Applications section of either document.

All subject matter of the Priority Applications and the Related Applications and of any and all parent, grandparent, great-grandparent, etc. applications of the Priority Applications and the Related Applications, including any priority claims, is incorporated herein by reference to the extent such subject matter is not inconsistent herewith.

### SUMMARY

In one aspect, a torso support includes at least one force applying element adapted to apply force to at least a portion of a torso of a subject wearing the torso support; at least one positioning element adapted to position the at least one force applying element with respect to the torso of the subject such that the at least one force applying element is located between the at least one positioning element and the torso of the subject and arranged to apply force to the at least a portion of the torso of the subject in a direction substantially normal to the surface of the at least a portion of the torso of the subject; electrical circuitry adapted to control the actuation of the at least one force applying element to apply force to the at least a portion of the torso of the subject, the electrical circuitry including: initiation circuitry adapted to generate a start signal; timing circuitry configured to control timing of actuation of the at least one force applying element by the electrical circuitry to: apply force during an application time period having an application duration responsive to receipt of a start signal; and release at least a portion of the applied force at the end of the application time period; a data storage location adapted to store the application duration. In addition to the foregoing, other system aspects are described in the claims, drawings, and text forming a part of the disclosure set forth herein.

In one aspect, a method of controlling an active torso support includes receiving an application duration from a data storage location on an active torso support; generating a start signal with initiation circuitry on the active torso support; applying force to at least a portion of a torso of a subject wearing the active torso support with at least one force applying element on the active torso support, responsive to the start signal and for an application time period having the application duration, the force being applied in a direction substantially normal to the surface of the at least a portion of the torso of the subject; and releasing at least a portion of the applied force from the at least a portion of the torso of the subject at the end of the application time period. In addition to the foregoing, other method aspects are described in the claims, drawings, and text forming a part of the disclosure set forth herein.

In one aspect, an article of manufacture includes one or more non-transitory machine-readable data storage media bearing one or more instructions for: receiving an application duration from a data storage location on an active torso support; generating a start signal with initiation circuitry on the active torso support; controlling at least one force applying element on the active torso support to apply force in a direction substantially normal to the surface of the at least a portion of the torso of the subject, responsive to the start signal and for an application time period having the application duration; and controlling the at least one force applying element to release at least a portion of the applied force from the at least a portion of the torso of the subject at the end of the application time period. In addition to the foregoing, other aspects of articles of manufacture including one or more non-transitory machine readable data storage media bearing one or more instructions are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

For a more complete understanding of embodiments, reference now is made to the following descriptions taken in connection with the accompanying drawings. The use of the same symbols in different drawings typically indicates similar or identical items, unless context dictates otherwise. The illustrative embodiments described in the detailed description, drawings, and claims are not meant to be limiting. Other embodiments may be utilized, and other changes may be made, without departing from the spirit or scope of the subject matter presented here.

FIG. 1 is a block diagram of active torso support.

FIG. 2 is an illustration of an embodiment of a torso support.

FIG. 3A illustrates an example of timing of force application by an active torso support.

FIG. 3B illustrates an example of timing of force application by an active torso support.

FIG. 4A illustrates an example of timing of force application by an active torso support.

FIG. 4B illustrates an example of timing of force application by an active torso support.

FIG. 5 is an illustration of a subject wearing an active torso support.

FIG. 6 illustrates an example of timing of force application by an active torso support.

FIG. 7 is a block diagram of an active torso support system.

FIGS. 8A and 8B are block diagrams portion of the torso support system of FIG. 7.

FIGS. 9A and 9B illustrate an example of timing of force application by an active torso support.

FIG. 10 is a flow diagram of a method of controlling an active torso support.

FIG. 11 is a flow diagram of a method of controlling an active torso support.

FIG. 12 is a flow diagram of a method of controlling an active torso support.

FIG. 13 is a flow diagram of a method of controlling an active torso support.

FIG. 14 is a flow diagram of a method of controlling an active torso support.

FIG. 15 is a flow diagram of a method of controlling an active torso support.

FIG. 16 is a flow diagram of a method of controlling an active torso support.

FIG. 17 is a flow diagram of a method of controlling an active torso support.

FIG. 18 illustrates an article of manufacture including non-transitory machine-readable data storage media bearing one or more instructions.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

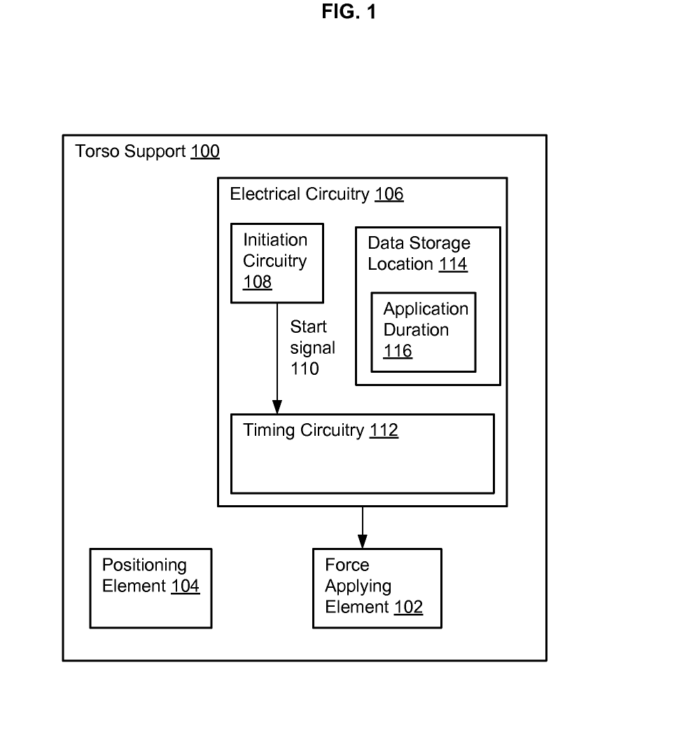

FIG. 1 is a block diagram of a generalized torso support system 100. Torso support 100 includes at least one force applying element 102 adapted to apply force to at least a portion of a torso of a subject wearing the torso support and at least one positioning element 104 adapted to position the at least one force applying element with respect to the torso of the subject such that the at least one force applying element is located between the at least one positioning element and the torso of the subject and arranged to apply force to the at least a portion of the torso of the subject in a direction substantially normal to the surface of the at least a portion of the torso of the subject. (The term “normal,” as used herein, has its usual geometric meaning, that is, a line or vector normal to a surface at a point is perpendicular to the tangent plane to the surface at that point.) Torso support 100 also includes electrical circuitry 106, which is adapted to control the actuation of the at least one force applying element 102 to apply force to the at least a portion of the torso of the subject. Electrical circuitry 106 includes initiation circuitry 108, which is adapted to generate a start signal 110; and timing circuitry 112. Timing circuitry 112 is configured to control timing of actuation of the at least one force applying element 102 by the electrical circuitry 106 to cause it to apply force during an application time period having an application duration responsive to receipt of a start signal; and release at least a portion of the applied force at the end of the application time period. Torso support 100 also includes a data storage location 114, which is adapted to store the application duration 116. In addition to the foregoing, other system aspects are described in the claims, drawings, and text forming a part of the disclosure set forth herein. In various aspects, the timing of application of force to the torso of the subject by the active torso support (e.g., as determined by the generation of start signal 110 and application duration 116) is controlled such that force is applied at the times when it is most beneficial to the subject. In various aspects, as discussed in greater detail herein below, in order to provide force application at a suitable time, a start signal is generated in response to one or more of a user input; gait, posture, or activity of the subject (including detected changes in gait, posture, or activity); a signal from a timing device (including a time-of-day signal, counter signal or timer-controlled interrupt), and other signal that are dependent on, correlated with, predictive of, or otherwise related to or indicate of a state of the subject in which activation of the torso support is or is expected to be beneficial.

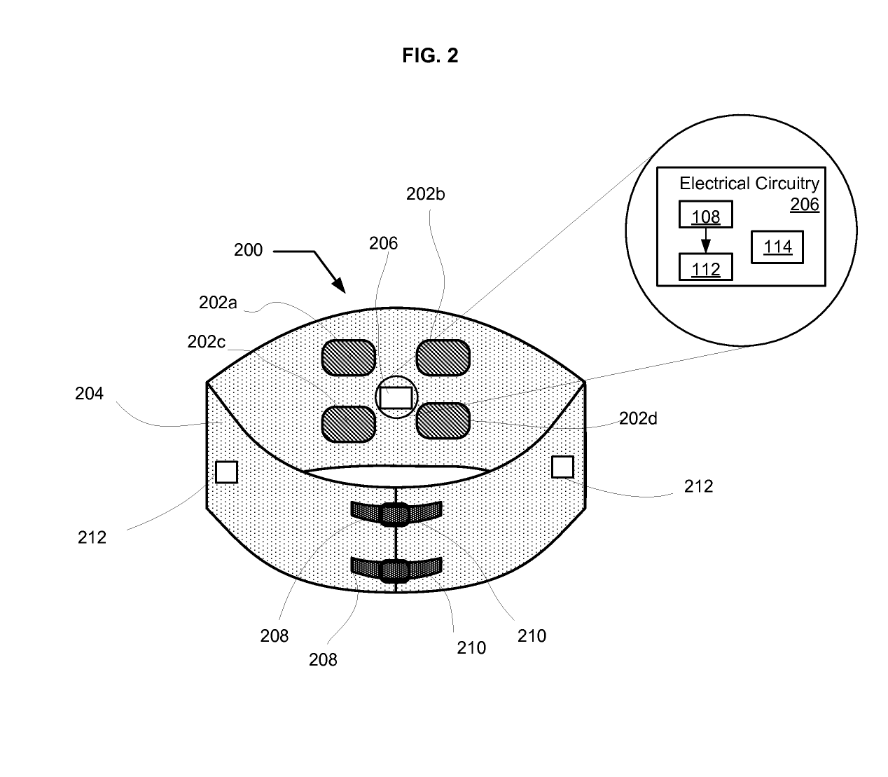

FIG. 2 depicts an example of an active torso support 200 configured as a back support or back brace. Force applying elements 202a-202d are positioned with respect to the torso of the subject by positioning element 204, which is configured as a belt adapted to be fitted around the waist/mid-torso of a subject. Force applying elements 202a-202d are expandable fluid/air filled bladders, for example as described in U.S. Pat. No. 4,135,503 to Romano; U.S. Pat. No. 6,540,707 to Stark et al, and U.S. Pat. No. 5,827,209 to Gross et al., each of which is incorporated herein by reference. Expansion of such bladders can be controlled through the use of a motorized pump and electrically controlled valves, with feedback provided by pressure sensors. Operation of active torso support 200 is controlled by electrical circuitry 206, which includes initiation circuitry 108, timing circuitry 112, and data storage location 114, as described in connection with FIG. 1. Positioning element 204 includes straps 208 and buckles 210 to secure positioning element 204 with respect to the torso of the subject. Torso support 200 also includes sensors 212.

In other aspects, a torso support as depicted generally in FIG. 1 can be configured to support or brace various portions of the torso, including, for example, one or more of all or portions of a side, an abdomen, a chest, a ribcage, a stomach, a hip, a pelvic region, an abdomen, a thorax, a shoulder, a buttock, a lower back, and an upper back.

A force applying element (e.g. force applying element 102 depicted in FIG. 1) can be any structure that is capable of applying force to a region of the torso of the subject, via a torso-contacting portion such as a pad or probe, and a controllable force-generating component that acts to move the torso contacting portion relative to the torso (e.g. by pressing against the torso and/or by applying shear forces to the torso, e.g. by engaging the surface of the torso by friction). A controllable force generating component can be controlled by electrical circuitry 106, e.g. via an electrical signal carried via an electrical connection or via a wireless signal such as an optical or electromagnetic signal transmitted from the control circuitry to the force applying element. Force applying element 102 may include one or more actuator, mechanical linkage, expandable element, inflatable element, pneumatic element, or hydraulic element, or other structures or components capable of applying force or pressure in a controlled fashion to a localized area of the torso.

A force applying element 102 can include a plate (which may be curved or planar) a probe, or any structure having shape and size suitable for applying force to a desired portion of the torso. A force applying element can also include a skin-engaging element adapted to apply tensile or shear force to the skin surface; for example a skin-engaging element may include an adhesive, suction cup, or a frictional surface, or other components known to those skilled in the art to provide for the application of tensile or shear forces to the skin.

In an aspect, the force applying element includes a passive force applying element and a controllable active force applying element. In an aspect, the force applying element has a controllable stiffness, a controllable dimension, and/or a controllable position relative to the positioning element. The force applying element can include one or more of a spring, an elastic material, or a viscoelastic material. In an aspect, the force applying element includes an actuator, which may include, for example, a mechanical linkage, an expandable element, an inflatable element, a screw, a pneumatic element, or a hydraulic element.

Force applying elements can be fluid/air filled bladders, as described above. In addition, Force applying elements can be mechanically or pneumatically driven force applying elements, e.g. as described in U.S. Pat. No. 5,624,383 to Hazard et al., which is incorporated herein by reference. Pneumatic and hydraulic piston type force applying elements as described in U.S. Pat. No. 6,746,413 to Reinecke et al., which is incorporated herein by reference, and screw thread/worm gear assembly structures as described in U.S. Published Patent Application 2009/0030359 to Wikenheiser et al., which is incorporated herein by reference, may be positioned to press against the torso (delivering force substantially perpendicular to the skin surface), or positioned apply shear forces (i.e., force having a significant component parallel to the skin surface).

Positioning element 104 can be any structure capable of holding force applying element 102 in position with regard to at least a portion of the torso of the subject, and may include, for example, at least one band, strap, belt, or harness, or a garment such as a corset, girdle, jacket, vest, or brief. The positioning element may include one or multiple straps or other components, without limitation. The positioning element can be constructed from flexible, resilient, or elastic material, including but not limited to leather, fabric, webbing, mesh, cable, cord, flexible metals or polymers, or sections of rigid metals, polymers or other materials connected in such a manner that the sections can be movably fitted around the torso of the subject, e.g. by a hinge or other linkage or by one or more sections of flexible material. Positioning element 104 may include fasteners to secure the positioning element with respect to the torso of the subject, e.g. straps and buckles as depicted in FIG. 2, or other fasteners as are known in the art, including but not limited to buckles, snaps, zippers, latches, clips, ties, hook and loop fasteners, lacings, and so forth. Positioning element may include an active or passive tensioning component (for example, elastic) to provide for tightening of the positioning element about the torso of the subject to provide for a secure fit. In an aspect, positioning element 104 simply includes an elastic component that allows it to be slid onto the torso of the subject, without the need for fasteners.

Force applying elements 102, control circuitry 106, and other system components described herein may be attached to the positioning element 104 or held in place by pressure or friction, e.g. by being pressed between the torso of the subject and the positioning element.

As noted above, electrical circuitry 106 in FIG. 1 includes initiation circuitry 108 and timing circuitry 112. Timing circuitry 112 is configured to control timing of actuation of the at least one force applying element 102. Under control of the electrical circuitry, the at least one force applying element 102 applies force to at least a portion of the torso of the subject during an application time period.

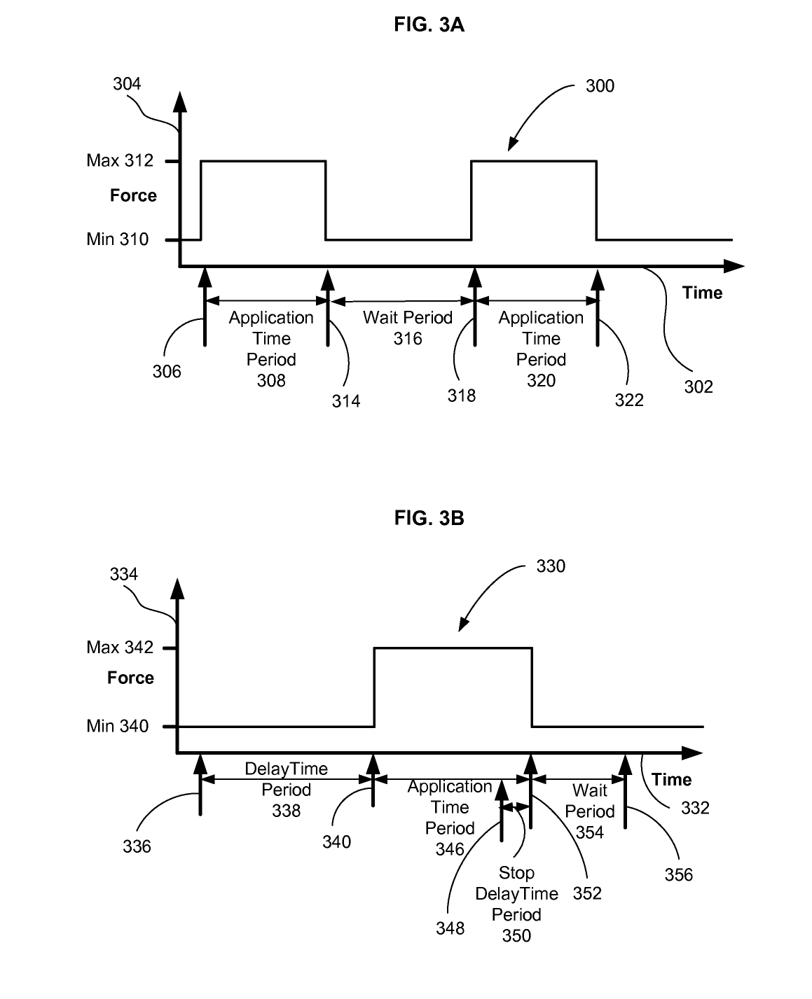

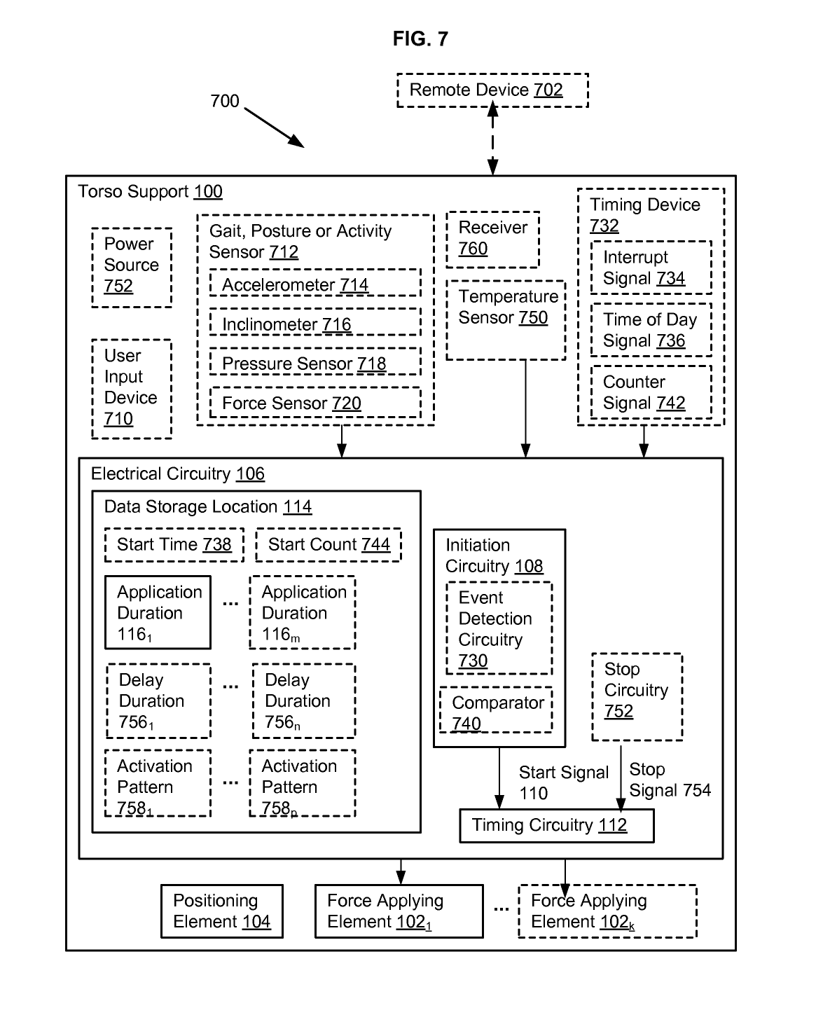

FIGS. 3A and 3B show examples of force applied by a force applying element of an active torso support as a function of time, with time represented on the horizontal axis and force on the vertical axis, and illustrate the terminology used herein to describe various parameters of force application. The timing and other aspects of force application depicted in FIGS. 3A and 3B are controlled by electrical circuitry as described generally in connection with FIG. 1, and described in greater detail herein below, e.g. in connection with FIG. 7. In FIG. 3A, trace 300 represents the force applied by a force applying element. Time is represented on horizontal axis 302 and the force is represented on vertical axis 304. Arrow 306 indicates the time at which a start signal is generated. In this example, application time period 308 starts substantially immediately in response to start signal 306. There is no delay between the generation of the start signal and start of the application time period. Application time period 308 has a duration that is determined by the application duration value (e.g., application duration 116 stored in data storage location 114). In the example shown in FIG. 3A, prior to generation of the start signal at 306, the applied force represented by trace 300 has a minimum value 310. During application time period 308, the applied force represented by trace 300 has a constant maximum force value 312. At the end of application time period 308, indicated by arrow 314, at least a portion of the applied force is released, and the force returns to minimum value 310, and is maintained at that level during wait period 316. The application time period can be repeated, with generation of a start signal at 318, followed by a second application time period 320, ending at 322. In FIG. 3B, trace 330 represents force applied by a force applying element. Time is represented on horizontal axis 332 and force is represented on vertical axis 334. Under control of electrical circuitry (e.g. electrical circuitry 106 in FIG. 1), following generation of a start signal at 336, there is a delay period 338, during which applied force is maintained at minimum force level 340, which is the same applied force level as prior to generation of the start signal. After delay period 338, the applied force is increased to the maximum force 342 at time 344, and this force level is maintained during application time period 346. In the example of FIG. 3B, a stop signal is generated at 348, and after a stop delay time period 350, application time period 344 ends and the applied force is decreased to minimum force level 340 at 352. A wait period 354 may occur between the end of application time period 344 and the generation of another start signal at 356. Depending on the nature of start signal 356, the duration of wait period 354 may be known a priori (e.g., in the case that start signal 356 is generated by a timing device) or unknown a priori (e.g., in the case that start signal 356 is generated in response to a sensed gait, posture or activity).

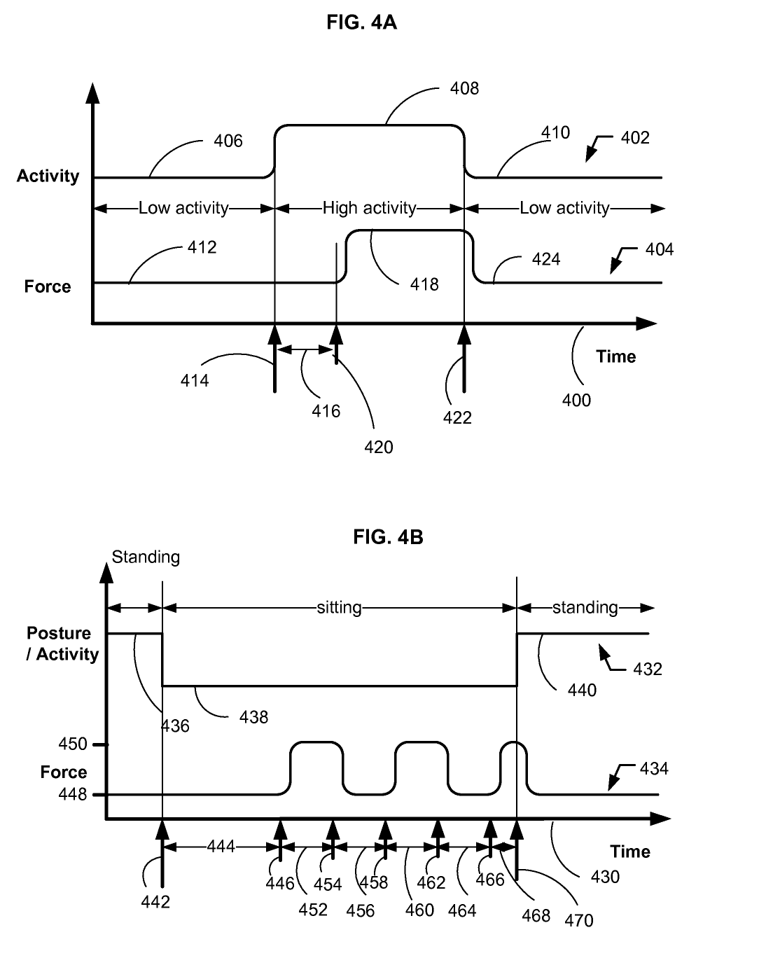

Further examples of force application as a function of time are depicted in FIGS. 4A and 4B. The timing and other aspects of force application depicted in FIGS. 4A and 4B are controlled by electrical circuitry as described generally in connection with FIG. 1, and described in greater detail herein below, e.g. in connection with FIG. 7. In FIG. 4A, time is represented on horizontal axis 400. Trace 402 represents the activity level of a subject wearing a torso support, which may be determined, for example, from a signal sensed by a sensor on the torso support. Trace 404 represents the force generated by the torso support, to provide support to the torso of the subject. The level of support provided by the torso support is varied in response to the activity level of the subject, which may be determined by various means, as described elsewhere herein. Trace 402 depicts a period of low activity 406, followed by a period of high activity 408, which is subsequently followed by a period of low activity 410. Such an activity pattern would be obtained, for example, if the subject went from a low activity state such as walking or standing, to a higher activity state such as lifting and carrying heavy boxes or performing some other physical task, and then returned to the lower activity state. It will be appreciated that “low activity” and “high activity” are relative terms, and may be defined differently for different subjects and/or for different applications. In the example of FIG. 4A, during the period of low activity 406, a lower level of force 412 is applied by the torso support. A start signal is generated at 414, responsive to detection of the change from low activity 406 to high activity 408. Under control of electrical circuitry (e.g. electrical circuitry 106 in FIG. 1), a delay time period 416 passes before applied force is increased to higher level 418, at time 420. In an aspect, delay time period 416 is selected in advance (by the subject or by a medical care provider, for example) such that support will be provided to the torso of the subject at or before the time the subject experiences weakness, soreness, or fatigue due to high activity. For example, if the subject knows from experience that after an hour of work he or she is likely to begin experiencing weakness, soreness, or fatigue, the subject might select a delay period of forty-five minutes so that extra support would be provided before the subject begins to experience symptoms. In the example of FIG. 4A, the higher level of force 418 is maintained until the activity of the subject is reduced to low activity level 410, at 422. In response to detection of the change from high activity 408 to low activity 410, the applied force is reduced to lower level 424.

While in many cases a subject may desire additional support to the torso during periods of high activity (such as manual labor), it will be appreciated that extended periods of low activity may also result in injury and/or discomfort, and application of force to provide support to portions of the torso during periods of low activity may also be beneficial. For example, extended periods of sitting may cause or exacerbate back pain. FIG. 4B depicts a pattern of force application in which a higher level of support is provided during a period of lower activity. In addition, FIG. 4B depicts a cyclical force application pattern. As before, time is represented on horizontal axis 430. The posture/activity of the subject is represented by trace 432, while the force applied by the torso support is represented by trace 434. As represented by trace 432, the subject is initially in a standing posture, then assumes a sitting posture, as indicated at 438, and eventually returns to a standing posture, as indicated at 440. A start signal is generated at 442, responsive to detection of the change from standing 436 to sitting 438. Following a delay time period 444, at 446, the applied force is increased from a lower level 448 to a higher level 450. Following application time period 452, at time 454, the applied force is reduced to lower level 448 for a wait period 456. At time 458, applied force is increased to higher level 450 and maintained at that level for application time period 460, which is of the same duration as application time period 452. At time 462, the applied force is again reduced to lower level 448 for a wait period 464. At time 466, applied force is again increased to higher level 450. However, application time period 468 is cut short, in that upon detection of the change in the subject's posture from sitting to standing, at 470, the applied force 434 is reduced to lower level 448, and is maintained at that level while the subject remains standing.

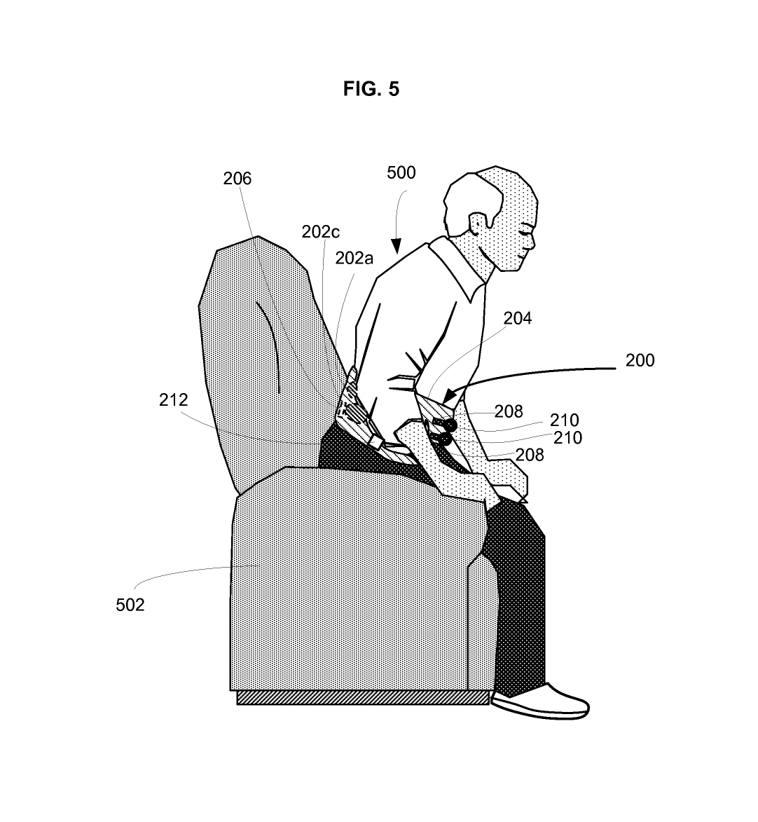

A temporal pattern of force application pattern such as that depicted in FIG. 4B can be applied, for example, by the system illustrated in FIG. 5. FIG. 5 depicts a subject 500 wearing a torso support 200, of the type depicted in FIG. 2. Torso support 200 provides support to the torso of subject 500 during activities such as sitting in chair 502, for example. Torso support 200 includes force applying elements (202a and 202c are visible in FIG. 5), and positioning element 204. Actuation of the force applying elements is controlled by electrical circuitry 206. Torso support 200 is secured on subject 500 with straps 208 and buckles 210, for example. The change in the posture of the subject from standing to sitting can be detected with one or more sensor 212 (only one sensor 212 is visible in FIG. 5, but torso support 200 may include two sensors, as shown in FIG. 2). Sensor(s) 212 may be, for example, an integrating accelerometer. Application of force to the torso of subject 500, according to a pattern as shown in FIG. 4B, may be initiated by detection of the change in posture of the subject from standing to sitting (as at 442 in FIG. 4B) and stopped when the subject rises from sitting to standing, e.g. as at 470 in FIG. 4B.

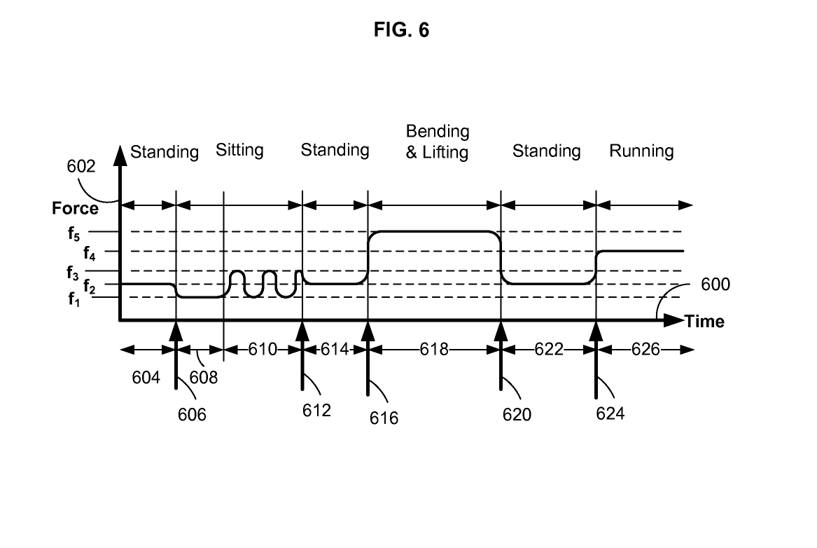

FIG. 6 depicts additional patterns of force application by an active torso support. In FIG. 6, time is represented on horizontal axis 600, and force is represented on vertical axis 602. Trace 604 represents the levels of force applied by a force applying element of the torso support during various activities. The various activities (standing, sitting, standing, bending & lifting, and running) are indicated along the top of FIG. 6, and transitions from one activity to another are indicated by heavy arrows on time axis 600. During a first time interval 604, the subject is standing, and a force f₂ is applied. At 606, the subject's activity changes from standing to sitting. The applied force is reduced to level f₁, but after a delay period 608, a cyclical force pattern is applied during interval 610 (in which the force is cyclically applied at level f₃, and then reduced to level f₁, generally as illustrated in FIG. 4B). At 612, the subject's posture/activity changes from sitting to standing, and the applied force is reduced to level f₂ and is maintained at the level over interval 614, for the duration that the subject is standing. At 616, the subject begins the activity of bending and lifting. Force is increased to level f₅ and maintained at this level for the duration 618 of this physically demanding task. At 620, the subject returns to standing, and force is decreased to level f₂ and maintained at this level over interval 622. At 624, the subject's activity changes to running, and force is increased to level f₄ and maintained at this level over interval 626, while the subject continues running It will be appreciated that the above activation pattern is merely an example, and the various choices of force level may be applied during different activities or postures. Furthermore, in various aspects, the applied forces and activation pattern(s) can be tailored to the needs of individual subjects.

FIG. 7 is a block diagram depicting components of a torso support system 700, including torso support 100 of the type described generally in connection with FIG. 1, which includes one or more force applying elements 102₁ . . . 102ₖ, (where k is the number of force applying elements), positioning element 104, electrical circuitry 106, initiation circuitry 108, and timing circuitry 112. Electrical circuitry 106 includes at least one data storage location 114, storing one or more application duration 116₁ . . . 116ₘ, (where m is the number of application duration values). In an aspect, torso support system 700 includes a remote device 702. Electrical circuitry 106 may include analog or digital circuitry electrical circuitry.

In a general sense, those skilled in the art will recognize that the various embodiments described herein can be implemented, individually and/or collectively, by various types of electrical circuitry having a wide range of electrical components such as hardware, software, firmware, and/or virtually any combination thereof. Electrical circuitry (including electrical circuitry 106 depicted in FIGS. 1 and 7, for example) includes electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of memory (e.g., random access, flash, read only, etc.)), electrical circuitry forming a communications device (e.g., a modem, communications switch, optical-electrical equipment, etc.), and/or any non-electrical analog thereto, such as optical or other analogs (e.g., graphene based circuitry). In a general sense, those skilled in the art will recognize that the various aspects described herein which can be implemented, individually and/or collectively, by a wide range of hardware, software, firmware, and/or any combination thereof can be viewed as being composed of various types of “electrical circuitry.”

Those skilled in the art will recognize that at least a portion of the devices and/or processes described herein can be integrated into a data processing system. Those having skill in the art will recognize that a data processing system generally includes one or more of a system unit housing, a video display, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

Force applying elements 102₁ . . . 102ₖ are as described in connection with FIG. 1, and are typically electromechanical in nature. It will be appreciated that a wide range of components may impart mechanical force or motion, such as rigid bodies, spring or torsional bodies, hydraulics, electro-magnetically actuated devices, and/or virtually any combination thereof. As used herein “electro-mechanical system” includes, but is not limited to, electrical circuitry operably coupled with a transducer (e.g., an actuator, a motor, a piezoelectric crystal, a Micro Electro Mechanical System (MEMS), etc). Those skilled in the art will recognize that electro-mechanical as used herein is not necessarily limited to a system that has both electrical and mechanical actuation except as context may dictate otherwise.

In an aspect, torso support 100 includes a user input device 710 operatively connected to the initiation circuitry 108, wherein the initiation circuitry 108 is adapted to generate start signal 110 responsive to an input from a user on the user input device 710.

In an aspect, torso support 100 includes at least one gait, posture, or activity sensor 712 operatively connected to initiation circuitry 108, wherein initiation circuitry 108 is adapted to generate start signal 110 responsive to a signal indicative of at least one of a gait, posture, or activity of the subject from the at least one gait, posture, or activity sensor. Gait, posture, or activity sensor 712 may include, for example, one or more of an accelerometer 714, inclinometer 716, pressure sensor 718, or force sensor 720. Detection of gait based on signals from accelerometers can be performed, for example, as described by Derawi et al., “Improved Cycle Detection for Accelerometer Based Gait Authentication,” IEEE Sixth International Conference on Intelligent Information Hiding and Multimedia Signal Processing,” Oct. 15-17, 2010, pp. 312-317; Sabelman et al., “Accelerometric Activity Identification for Remote Assessment of Quality of Movement”, Proceedings of the 26^{th} Annual International Conference of the IEEE EMBS, San Francisco, Calif., USA, Sep. 1-5, 2005, pp. 4781-4784; Rong et al., “A Wearable Acceleration Sensor System for Gait Recognition,” 2007 Second IEEE Conference on Industrial Electronics and Applications, May 23-25, 2007, pp. 2654-2659; and Sekine et al., “Discrimination of Walking Patterns Using Wavelet-Based Fractal Analysis,” IEEE Transactions on Neural Systems and Rehabilitation Engineering, Vol. 10, No. 3, September 2002, pp. 188-196, each of which is incorporated herein by reference. The torso support can include other types of sensors, including but not limited to gyro sensors (e.g., to indicate inclination or leaning over of the subject), magnetometers (which provide angle information, or can be used with external field coils to provide both position and angle), differential position sensors (using GPS or pseudo-GPS signals), inclinometers (e.g., a MEMS type digital inclinometer such as an Analog Devices ADIS16209) that can be used to detect the inclination of the subject's torso. An inclinometer can be used in combination with other sensors to provide information regarding the angular position of the subject's limbs or spine, which is indicative of aspects of the subjects gait, and may also provide information regarding disturbances in gait, including tilting, swaying or falling. A torso support can include one or multiple sensors, without limitation.

In an aspect, one or more sensors can be operably coupled to torso support 100 but located at a distance from the torso support, on the body or remote from the body. For example, accelerometers located on various portions of the body can be used to provide signals indicative of the gait of the subject, including on the legs (see, e.g. Torrealba et al., “Statistics-based technique for automated detection of gait events from accelerometer signals,” Electronics Letters, 28 Oct. 2010, Vol. 46, No. 22, and Itoh et al., “Development of New Instrument for Evaluating Leg Motions Using Acceleration Sensors,” Environmental Health and Preventive Medicine 12, 111-118, May 2007, each of which is incorporated herein by reference), legs and/or arms (see Mannini et al., “Accelerometry-Based Classification of Human Activities Using Markov Modeling,” Computational Intelligence and Neuroscience, Vol. 2011, Article ID 647858, published online 4 Sep. 2011, which is incorporated herein by reference), and/or head (see Sabelman et al., “Accelerometric Activity Identification for Remote Assessment of Quality of Movement”, Proceedings of the 26^{th} Annual International Conference of the IEEE EMBS, San Francisco, Calif., USA, Sep. 1-5, 2005, pp. 4781-4784, which is incorporated herein by reference).

In an aspect, initiation circuitry 108 includes event detection circuitry 730, which is adapted to process the signal from the at least one gait, posture, or activity sensor 712 to detect a change in the gait, posture or activity of the subject, and wherein initiation circuitry 108 is adapted to generate start signal 110 responsive to detection of the change in gait, posture or activity of the subject by the event detection circuitry 730. Processing of the signal by event detection circuitry can include, for example, processing of signals from accelerometers worn on a subject's hip, wrist, arm, ankle and thigh to distinguish a variety of activities, including walking, running, standing, and climbing stairs (see Mannini et al., “Accelerometry-Based Classification of Human Activities Using Markov Modeling,” Computational Intelligence and Neuroscience, Vol. 2011, Article ID 647858, published online 4 Sep. 2011, and Sekine et al., “Discrimination of Walking Patterns Using Wavelet-Based Fractal Analysis,” IEEE Transactions on Neural Systems and Rehabilitation Engineering, Vol. 10, No. 3, September 2002, pp. 188-196, each of which is incorporated herein by reference), or processing of data from accelerometers located on the hips of a subject to distinguish walking, turning, ascending or descending stairs, as described in Sabelman et al., (“Accelerometric Activity Identification for Remote Assessment of Quality of Movement”, Proceedings of the 26^{th} Annual International Conference of the IEEE EMBS, San Francisco, Calif., USA, Sep. 1-5, 2005, pp. 4781-4784), which is incorporated herein by reference.

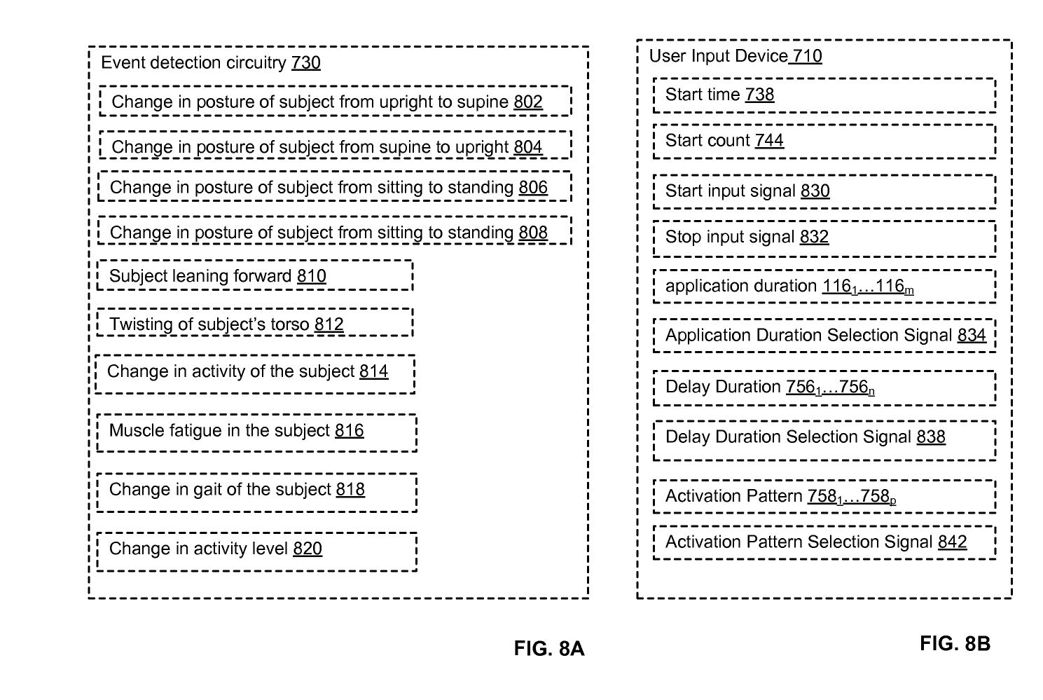

As shown in FIG. 8A, in an aspect event detection circuitry 730 may be adapted to process the signal from the at least one gait, posture, or activity sensor to detect various changes in the posture of the subject, a change in the posture of the subject from upright to supine, as indicated at 802, supine to upright, as indicated at 804, sitting to standing, as indicated at 806, or from standing to sitting, as indicated at 808. In an aspect, event detection circuitry 730 is adapted to process the signal from the at least one gait, posture, or activity sensor to detect the subject leaning forward, as indicated at 810, to detect twisting of the subject's torso, as indicated at 812, to detect a change in the activity of the subject, as indicated at 814, to detect muscle fatigue in the subject, as indicated at 816, to detect a change in the gait of the subject, as indicated at 818, or to detect a change in an activity level, as indicated at 820.

Referring back to FIG. 7, in an aspect, torso support 100 includes a timing device 732 operatively connected to initiation circuitry 108, wherein initiation circuitry 108 is adapted to generate start signal 110 responsive to receipt of a signal from timing device 732. In an aspect, timing device 732 is configured to generate an interrupt signal 734, and initiation circuitry 108 is adapted to generate the start signal responsive to receipt of the interrupt signal 734. In an aspect, timing device 732 is configured to generate a time-of-day signal 736, wherein data storage location 114 is adapted to store a start time 738, wherein initiation circuitry 108 includes comparator 740 configured to compare the time-of-day signal 736 to start time 738, and wherein initiation circuitry 108 is configured to generate start signal 110 based on a comparison of time-of-day signal 736 to start time 738. In an aspect, torso support 100 includes user input device 710, which is operatively connected to electrical circuitry 106, wherein electrical circuitry 106 is adapted to receive start time 738 from a user via user input device 710 and store start time 738 in data storage location 114. In an aspect, timing device 732 is configured to generate a counter signal 742, wherein data storage location 114 is adapted to store a start count 744, wherein initiation circuitry 108 includes comparator 740 configured to compare counter signal 742 to start count 744, and wherein initiation circuitry 108 is configured to generate start signal 110 based on a comparison of counter signal 742 to start count 744. In an aspect, electrical circuitry 106 is adapted to receive a signal indicative of start count 744 from the user via the user input device 710 and store start count 744 in data storage location 114. Timing and counter devices, and their programming and operation, are well known to those having skill in the electronic arts.

In an aspect, torso support 100 includes at least one temperature sensor 750, which is operatively connection to electrical circuitry 106, wherein electrical circuitry 106 is configured to control the actuation of at least one force applying element 102₁ . . . 102ₖ to apply force to the at least a portion of the torso of the subject based at least in part upon the temperature sensed by temperature sensor 750, which may be, for example, a semiconductor temperature sensor, thermistor, thermocouple, resistance-based temperature sensor, or an infrared temperature sensor. Temperature sensor 750 may be configured (based on type and positioning of the sensor) to sense the body temperature of the subject, the ambient temperature, or a combination thereof.

In an aspect, torso support 100 includes a power source 752, which may be battery or any of various types of power sources known to those skilled in the art. In an aspect, power source 752 is operatively connected to initiation circuitry 108, and initiation circuitry 108 is adapted to generate start signal 110 responsive to receipt of a power signal from power source 752.

In an aspect, torso support 100 includes a receiver 760, which is adapted to receive one or more application duration 116₁ . . . 116ₘ from remote device 702 via a wireless connection, wherein electrical circuitry 106 is adapted to store the one or more application duration 116₁ . . . 116ₘ received from the remote device 702 in data storage location 114. A wireless connection may occur via electromagnetic, radiofrequency, optical, infrared, or acoustic signals. Wireless communication between one or more local and remote devices is well known. See, e.g. U.S. Pat. No. 8,170,656 issued May 1, 2012, to Tan et al., and U.S. Published Application No. 2010/0198067 to Mahfouze et el., dated Aug. 5, 2010, each of which is incorporated herein by reference. Wireless communication may take place within a variety of networks, including but not limited to cellular network, local area network (LAN), wireless local area network (WLAN), body area network (BAN), Bluetooth, and ZigBee. Remote device 702 may comprise a computing device or memory with stored data or instructions. Remote device 702 may comprise a user input device such as a cell phone or personal data assistant, a desktop, notebook or tablet computer, or the like.

In an aspect, torso support 100 includes user input device 710 operatively connected to electrical circuitry 106. In connection therewith, electrical circuitry 106 is adapted to receive one or more application duration 116₁ . . . 116ₘ from a user via user input device 710, and store one or more application duration 116ₖ . . . 116ₘ in data storage location 114. User input device 710 can be any of a variety of user input devices, as known to those of skill in the art, including but not limited to, a keyboard, keypad, mouse, or microphone, for example. While a single input device is discussed herein, it will be appreciated that system 700 may include multiple input devices of the same or different types, without limitation. Information provided to electrical circuitry via user input device 710 may include a start time 738, a start count 744, and/or one or more application durations 116₁ . . . 116ₘ, for example.

As shown in FIG. 7, in an aspect, data storage location 114 of torso support 100 is adapted to store a plurality of application durations 116₁ . . . 116ₘ, and electrical circuitry 106 is adapted to select the application duration from the plurality of application durations 116₁ . . . 116ₘ, stored in the data storage location 114 based on the application duration selection signal. Electrical circuitry 106 is adapted to receive an application duration selection signal from the user via the user input device 710.

FIG. 8B depicts examples of various inputs that can be entered via user input device 710 and provided to electrical circuitry (e.g. electrical circuitry 106 in FIG. 7). Inputs that can be entered via user input device 710 include, but are not limited to, start input signal 830, stop input signal 832, start time 738, start count 744, application durations 116₁ . . . 116ₘ, and application duration selection signal 834, for example.

Returning to FIG. 7, in another aspect, torso support 100 includes at least one sensor operatively connected to the electrical circuitry (e.g. gait, posture or activity sensor 712, or temperature sensor 750, for example), wherein data storage location 114 is adapted to store a plurality of application durations 116₁ . . . 116ₘ and wherein the electrical circuitry 106 is adapted to receive a sense signal from the sensor (712 or 750) and select the application duration from the plurality of application durations 116₁ . . . 116ₘ stored in data storage location 114 based on the sense signal.

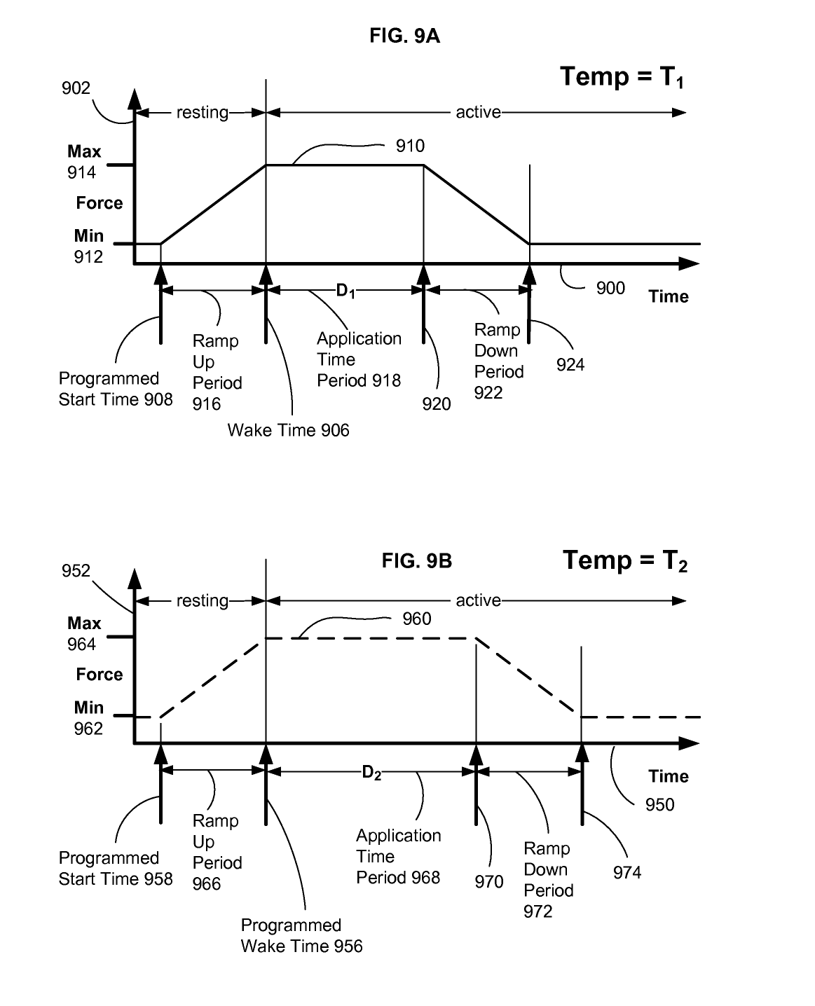

An example of an embodiment in which the application duration is selected based on a sense signal is described in connection with FIGS. 9A and 9B. Each of FIGS. 9A and 9B depicts a pattern of activation of a torso support used to provide support to the torso of a subject as the subject is transitioning from a resting activity state to an active activity state, e.g. as the subject is rising from bed in the morning. In the embodiment of FIGS. 9A and 9B, the application duration is selected based upon a sensed temperature. The temperature can be sensed by a sensor on the torso support that is configured to select the ambient temperature, the body temperature of the subject, or a combination thereof. It is thought that if the sensed temperature is lower, the subject will experience greater muscle stiffness and discomfort, and it is desirable to provide support to the torso of the subject for a longer amount of time, as it will take more time for the subject's body to warm up sufficiently to enable safe and comfortable movement and flexibility. FIG. 9A depicts a pattern of activation in which the sensed temperature is T₁, and an application duration D₁ is selected. FIG. 9B depicts a pattern of activation in which the sensed temperature is T₂, and an application duration D₂ is selected. In the example shown in FIGS. 9A and 9B, T₁, >T₂ and accordingly, D₁<D₂. In FIG. 9A, time is represented on horizontal axis 900, and force applied by one or more force applying element of a torso support is represented on vertical axis 902. Heavy arrows below horizontal axis 900 indicate time-related events. In particular, wake time is indicated at 906. As can be seen, wake time 906 corresponds to the time at which the subject's activity state changes (or is expected to change) from resting to active. In the present example, it is assumed that the wake time of the subject is predictable in advance. For example, wake time 906 can be the time at which the subject's alarm clock is scheduled to go off, or the time at which the subject typically wakes up. Start time 908 for activation of the torso support is programmed to occur at a fixed time prior to wake time 906. At start time 908, the force applied by the torso support (represented by trace 910) is increased under the control of electrical circuitry on the torso support from minimum force level 912 to maximum force value 914, over the course of ramp-up period 916, so that at wake time 906, the applied force 910 has reached its maximum force value 914. Applied force 910 is maintained at maximum force value 914 over the course of application time period 918, which has an application duration D₁. At 920, force begins to decrease from maximum force level 914 to minimum force level 912 over ramp-down period 922. By 924, it is presumed that the subject's muscles are sufficiently warmed up that applied force 910 at minimum force level 912 is sufficient for the subject's needs.

In FIG. 9B, time is represented on horizontal axis 950, and force applied by one or more force applying element of a torso support is represented on vertical axis 952. Wake time is indicated at 956. As in FIG. 9A, start time 958 for activation of the torso support is programmed to occur at a fixed time prior to wake time 956. At start time 958, the force applied by the torso support (represented by trace 960) is increased under the control of electrical circuitry on the torso support from minimum force level 962 to maximum force value 964, over the course of ramp-up period 966, so that at wake time 956, the applied force 960 has reached its maximum force value 964. Applied force 960 is maintained at maximum force value 964 over the course of application time period 968, which has an application duration D₂. At 970, force begins to decrease from maximum force level 964 to minimum force level 962 over ramp-down period 972. By 974, it is presumed that the subject's muscles are sufficiently warmed up that applied force 960 at minimum force level 962 is sufficient for the subject's needs. Selection of application duration based on sensed temperature can be accomplished by comparing the sensed temperature to a table of temperature values, and selecting the application duration corresponding to a particular temperature value (e.g., by matching the sensed temperature value with a value in a table, or by determining whether the sensed temperature value is within a range of temperature values specified by data contained in the table.

Returning to FIG. 7, in an aspect, torso support 100 includes stop circuitry 752 adapted to generate a stop signal 754, wherein electrical circuitry 106 is adapted to control actuation of at least one force applying element 102₁ . . . 102ₖ to release at least a portion of the force applied to the at least a portion of the torso of the subject by the at least one force applying element 102₁ . . . 102ₖ responsive to stop signal 754. In an aspect, at least one sensor (e.g. gait, posture or activity sensor 712, or temperature sensor 750, for example) is operatively connected to stop circuitry 752, wherein stop circuitry 752 is adapted to generate the stop signal 754 in response to a sense signal from the sensor. For example, as depicted in FIG. 4A, a stop signal may be generated in response to detection of a change in activity of the subject from a high activity level to a lower activity level at 422. In FIG. 4B, a stop signal may be generated in response to detection of a change in the subject's posture from sitting to standing, at 470. As a further example, the activation scheme depicted in FIGS. 9A and 9B could be modified such that a stop signal could be based at least in part on the sensed signal.

In an aspect, user input device 710 is operatively connected to stop circuitry 752, wherein stop circuitry 752 is adapted to generate stop signal 754 responsive to an input from a user on the user input device 710. For example, a user may wish to discontinue force application by the torso support for various reasons, such as comfort, safety, or convenience, to change or override a programmed activation pattern, etc. In an aspect, torso support 100 includes timing circuitry 112 that is configured to control timing of actuation of the at least one force applying element 102₁ . . . 102ₖ to begin the application time period after a delay time period following receipt of the start signal, the delay time period having a delay duration. Beginning the application time period after a delay time period in this manner is illustrated, for example, in FIGS. 3B, 4A, and 4B. In order to implement such control with a system as shown in FIG. 7, data storage location 114 is adapted to store one or more delay duration 756₁ . . . 756ₙ. In an aspect, torso support 100 includes receiver 760, which is adapted to receive the delay duration 756₁ . . . 756ₙ from a remote device 702 via a wireless connection, wherein electrical circuitry 106 is adapted to store one or more delay duration 756₁ . . . 756ₙ received from remote device 702 in data storage location 114. In an aspect, torso support 100 includes user input device 710 operatively connected to electrical circuitry 106, wherein electrical circuitry 106 is adapted to receive one or more delay duration 756₁ . . . 756ₙ from the user via user input device 710 and store the one or more delay duration 756₁ . . . 756ₙ d in data storage location 114. In an aspect, data storage location 114 is adapted to store a plurality of delay durations 756₁ . . . 756ₙ, and electrical circuitry 106 is adapted to receive a delay duration selection signal from the user via the user input device 710 and select the delay duration from the plurality of delay durations stored in the data storage location based on the delay duration selection signal. As shown in FIG. 8B, one or more delay durations 756₁ . . . 756ₙ, and delay duration selection signal 838 are among the various inputs that can be provided by a user via user input device 710.

Returning to FIG. 7, in an aspect, torso support 100 includes at least one sensor (e.g. gait, posture, or activity sensor 712 or temperature sensor 750, as described herein above) operatively connected to electrical circuitry 106, data storage location 114 is adapted to store a plurality of delay durations 756₁ . . . 756ₙ, and electrical circuitry 106 is adapted to receive a sense signal from the sensor and select the delay duration from the plurality of delay durations 756₁ . . . 756ₙ stored in data storage location 114 based on the sense signal.

In an aspect, electrical circuitry 106 is adapted to control the actuation of the force applying elements 102₁ . . . 102ₖ, to apply force to the at least a portion of the torso of the subject during the application time period according to an activation pattern 758₁ . . . 758ₚ. Various examples of activation patterns are depicted in the figures herein above. For example, FIGS. 3A and 3B depict simple activation patterns in which a force of constant amplitude is applied during the application time period. FIG. 4A depicts an activation pattern in which a constant force is applied for most of the application time period, but the transition between force levels is gradual, rather than step-wise as in FIGS. 3A and 3B. FIG. 4B depicts a cyclical activation pattern. FIGS. 9A and 9B depict activation patterns in which the activation time period is preceded by a ramp-up period and followed by a ramp-down period. It will be appreciated that an application pattern can specify force amplitudes and durations of one or more phases of the application time period, specify a function for calculating force amplitude as a function of time, or specify force amplitude at selected time intervals during the application time period. The latter approaches may be suitable for specifying complex application patterns, for example.

In an aspect, data storage location 114 is adapted to store activation pattern 758₁ . . . 758ₚ. In an aspect, torso support 100 includes a user input device 710 operatively connected to electrical circuitry 106, wherein electrical circuitry 106 is adapted to receive the activation pattern 758₁ . . . 758ₚ from the user via the user input device 710 and store the activation pattern 758₁ . . . 758ₚ in the data storage location 114. In an aspect, data storage location 114 is adapted to store a plurality of activation patterns 758₁ . . . 758ₚ, and wherein the electrical circuitry is adapted to control the actuation of the at least one force applying element 102₁ . . . 102ₖ to apply force to the at least a portion of the torso of the subject during the application time period according to an activation pattern selected from the plurality of activation patterns 758₁ . . . 758ₚ.

In an aspect, torso support 100 includes user input device 710 operatively connected to the electrical circuitry 106, wherein electrical circuitry 106 is adapted to receive an activation pattern selection signal from a user via the user input device, and to select the activation pattern from the plurality of activation patterns 758₁ . . . 758ₚ based on the activation pattern selection signal. As shown in FIG. 8B, activation patterns 758₁ . . . 758ₚ, and/or activation pattern selection signal 842 are among the various inputs that can be provided by a user via user input device 710.

Alternatively, or in addition, in an aspect, torso support 100 includes at least one sensor (e.g. gait, posture, or activity sensor 712, or temperature sensor 750) operatively connected to electrical circuitry 106, and electrical circuitry 106 is adapted to receive a sense signal from the at least one sensor, and to select the activation pattern from the plurality of activation patterns 758₁ . . . 758ₚ based on the sense signal. The plurality of activation patterns 758₁ . . . 758ₚ can include patterns corresponding to a plurality of pre-defined gaits, postures, or activities of the subject. For example, the plurality of pre-defined gaits, postures, or activities of the subject can include at least one of standing, sitting, lying, walking, standing up, sitting down, twisting, leaning forward, or lying down.

In an aspect, timing circuitry 112 is configured to control timing of actuation of the at least one force applying element 102₁ . . . 102ₖ by the electrical circuitry to produce cyclical activation and deactivation of the at least one force applying element. For example, the timing circuitry may include hardware and/or software capable of generating a cyclical signal, for example as depicted in FIG. 4B, by a variety of approaches known to those having skill in the electronic arts. For example, a cyclical signal can be generated under software control, through the use of software loop, or in response to detection of signals from a timing device (e.g. timing device 732). In addition, construction of hardware based circuits capable of producing cyclical signals is well known.

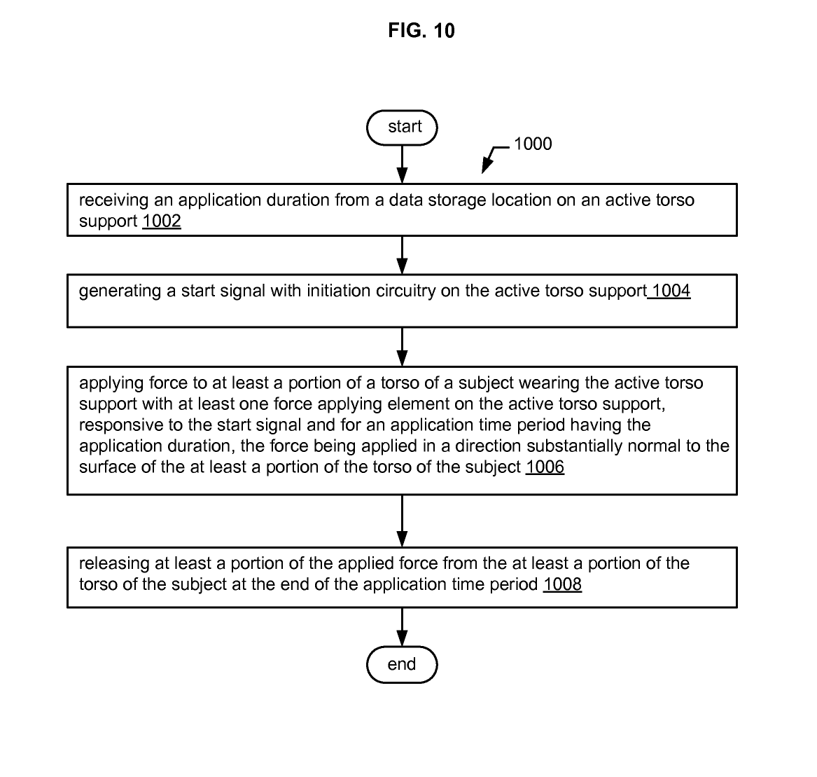

FIG. 10 depicts a method of controlling an active torso support. Method 1000 includes receiving an application duration from a data storage location on an active torso support 1002; generating a start signal with initiation circuitry on the active torso support 1004; applying force to at least a portion of a torso of a subject wearing the active torso support with at least one force applying element on the active torso support, responsive to the start signal and for an application time period having the application duration, the force being applied in a direction substantially normal to the surface of the at least a portion of the torso of the subject 1006; and releasing at least a portion of the applied force from the at least a portion of the torso of the subject at the end of the application time period 1008.

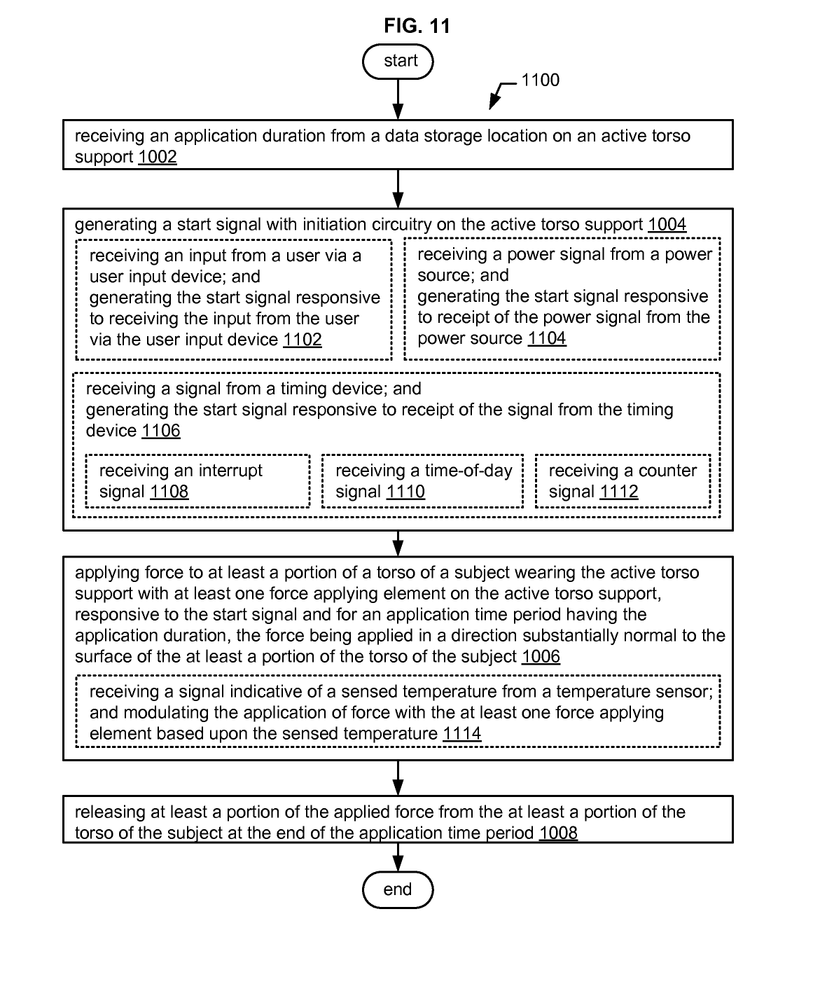

FIGS. 11-17 depict variations and expansions of method 1000 as shown in FIG. 10. In the methods depicted in FIGS. 11-17, steps 1002-1008 are as described generally in connection with FIG. 10. Method steps outlined with dashed lines represent steps that are included in some, but not all method aspects, and combinations of steps other than those specifically depicted in the figures are possible as would be known by those having ordinary skill in the relevant art.

In an aspect, as shown in FIG. 11, a method 1100 of controlling an active torso support includes receiving an input from a user via a user input device; and generating the start signal responsive to receiving the input from the user via the user input device, as indicated at 1102. In another aspect, method 1100 includes receiving a power signal from a power source; and generating the start signal responsive to receipt of the power signal from the power source, as indicated at 1104.

In an aspect, a method 1100 includes receiving a signal from a timing device; and generating the start signal responsive to receipt of the signal from the timing device, as indicated at 1106. In an aspect, receiving the signal from the timing device includes a receiving an interrupt signal 1108. In another aspect, receiving the signal from the timing device includes a receiving a time-of-day signal, as indicated at 1110. In another aspect, receiving the signal from the timing device includes a receiving a counter signal, as indicated at 1112.

In an aspect, a method 1100 includes receiving a signal indicative of a sensed temperature from a temperature sensor; and modulating the application of force with the at least one force applying element based upon the sensed temperature, as indicated at 1114.

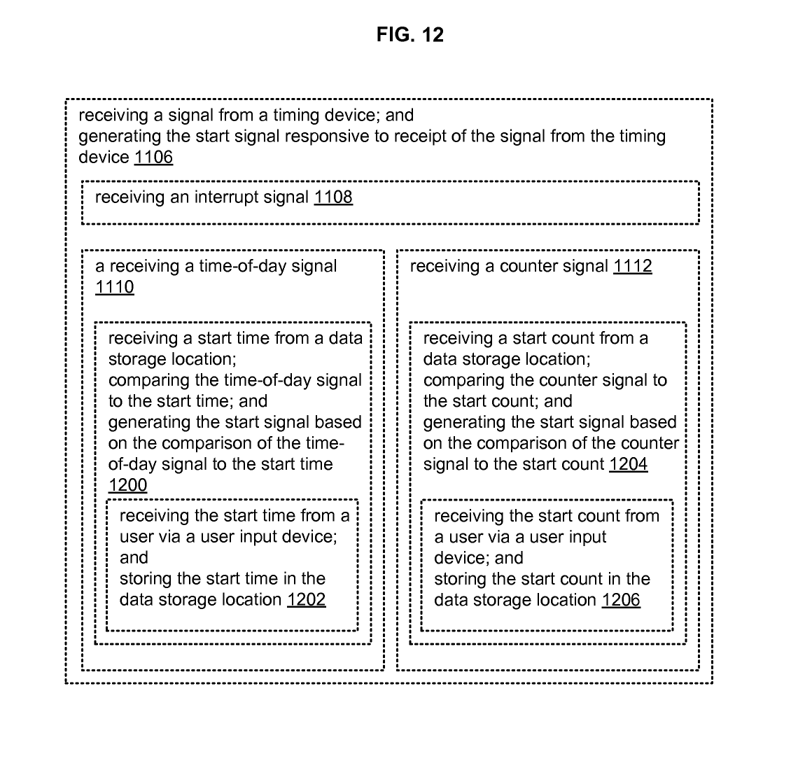

Further detail the method 1100 is shown in FIG. 12. In addition to receiving a signal from a timing device; and generating the start signal responsive to receipt of the signal from the timing device at 1106, and receiving a time-of-day signal 1110, in an aspect, the method further includes receiving a start time from a data storage location; comparing the time-of-day signal to the start time; and generating the start signal based on the comparison of the time-of-day signal to the start time, as indicated at 1200. The method may further include receiving the start time from a user via a user input device; and storing the start time in the data storage location, as indicated at 1202.

In another aspect, shown in FIG. 12, in addition to receiving a signal from a timing device and generating the start signal responsive to receipt of the signal from the timing device at 1106, and receiving a counter signal 1112, a method includes receiving a start count from a data storage location; comparing the counter signal to the start count; and generating the start signal based on the comparison of the counter signal to the start count, as indicated at 1204. The method may then also include receiving the start count from a user via a user input device; and storing the start count in the data storage location, as indicated at 1206.

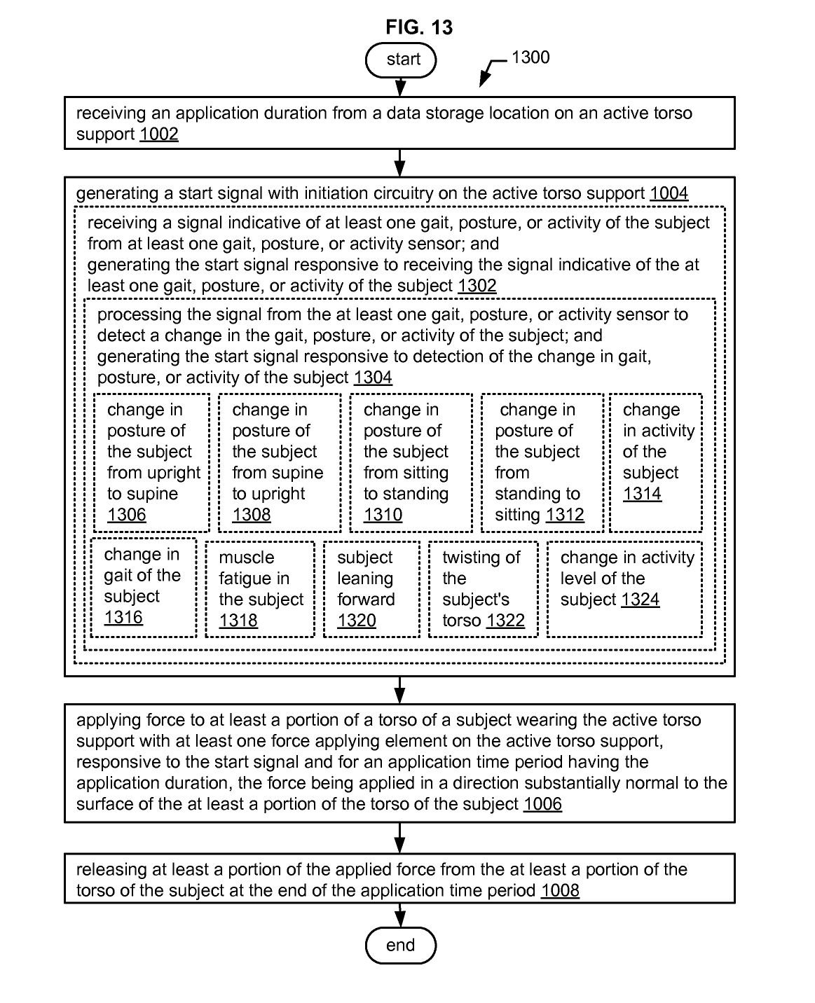

In an aspect, as shown in FIG. 13, a method 1300 of controlling an active torso support includes receiving a signal indicative of at least one gait, posture, or activity of the subject from at least one gait, posture, or activity sensor; and generating the start signal responsive to receiving the signal indicative of the at least one gait, posture, or activity of the subject, as indicated at 1302. Generating the start signal then includes processing the signal from the at least one gait, posture, or activity sensor to detect a change in the gait, posture, or activity of the subject, and generating the start signal responsive to detection of the change in gait, posture, or activity of the subject as indicated at 1304. For example, generating the start signal can include generating the start signal in response to detection of a change in posture of the subject from upright to supine 1306, from supine to upright 1308, from sitting to standing 1310, or from standing to sitting 1312, or generating the start signal in response to detection of a change in activity of the subject 1314, change in gait of the subject 1316, muscle fatigue in the subject 1318, the subject leaning forward 1329, twisting of the subject's torso 1322, or change in activity level of the subject 1324.

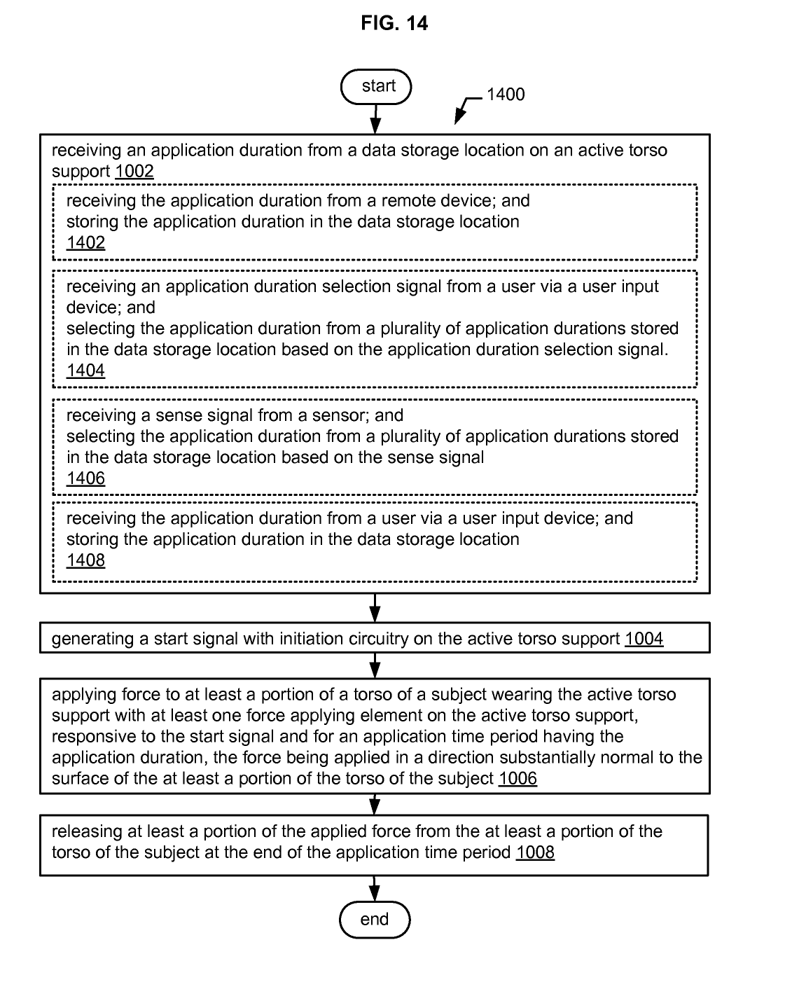

The application duration, that is, the duration of the application time period, can be determined in a number of ways. One or more of the following approaches can be employed in a given embodiment. As shown in FIG. 14, a method 1400 of controlling an active torso support includes receiving the application duration from a remote device; and storing the application duration in the data storage location, as indicated at 1402.

In one aspect, method 1400 includes receiving an application duration selection signal from a user via a user input device; and selecting the application duration from a plurality of application durations stored in the data storage location based on the application duration selection signal, as indicated at 1404.

In one aspect, method 1400 includes receiving a sense signal from a sensor; and selecting the application duration from a plurality of application durations stored in the data storage location based on the sense signal, as indicated at 1406.

In yet another aspect, method 1400 includes receiving the application duration from a user via a user input device; and storing the application duration in the data storage location, as indicated at 1408.

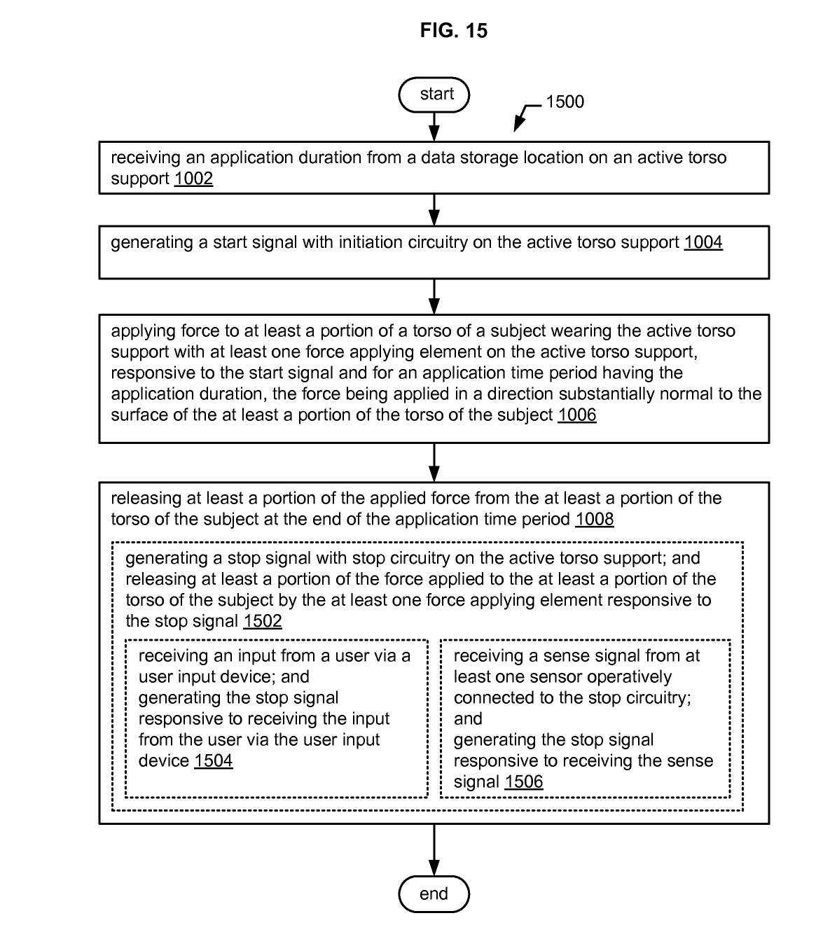

FIG. 15 illustrates a method 1500 of controlling an active torso support, including generating a stop signal with stop circuitry on the active torso support; and releasing at least a portion of the force applied to the at least a portion of the torso of the subject by the at least one force applying element responsive to the stop signal, as indicated at 1502. In an aspect, method 1500 includes receiving an input from a user via a user input device; and generating the stop signal responsive to receiving the input from the user via the user input device, as indicated at 1504. In another aspect, method 1500 includes receiving a sense signal from at least one sensor operatively connected to the stop circuitry; and generating the stop signal responsive to receiving the sense signal, as indicated at 1506.

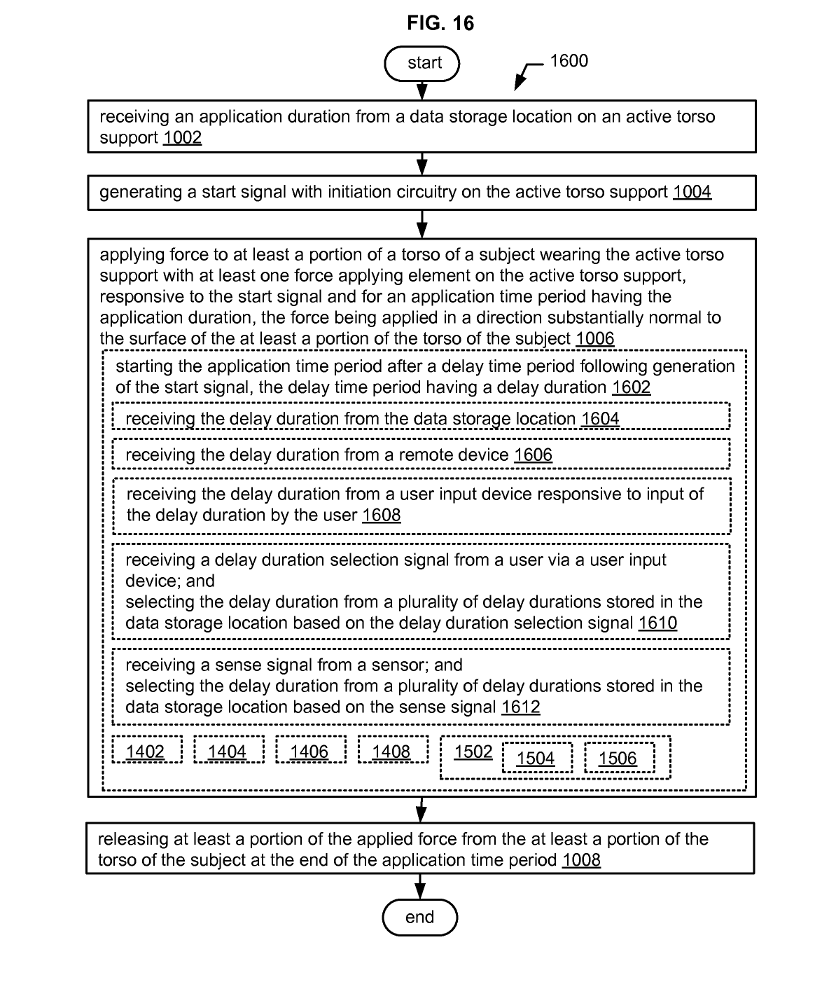

FIG. 16 depicts a method 1600, which includes starting the application time period after a delay time period following generation of the start signal, the delay time period having a delay duration, at 1602. In an aspect, method 1600 includes receiving the delay duration from the data storage location, at 1604, receiving the delay duration from a remote device, at 1606, or receiving the delay duration from a user input device responsive to input of the delay duration by the user, at 1608.

In another aspect, the delay duration can be selected from a plurality of delay durations stored in the data storage location, based on a user input or other input. For example, in an aspect, method 1600 includes receiving a delay duration selection signal from a user via a user input device; and selecting the delay duration from a plurality of delay durations stored in the data storage location based on the delay duration selection signal, as indicated at 1610. In a further aspect, method 1600 includes receiving a sense signal from a sensor; and selecting the delay duration from a plurality of delay durations stored in the data storage location based on the sense signal, at 1612.

In addition, the application duration be used in combination with the delay duration may be received and/or selected by various methods, as described elsewhere herein. For example, method 1600 may also include receiving the application duration from a remote device; and storing the application duration in the data storage location, as indicated at 1402 (described in connection with FIG. 14), or receiving an application duration selection signal from a user via a user input device; and selecting the application duration from a plurality of application durations stored in the data storage location based on the application duration selection signal, as indicated at 1404 (described in connection with FIG. 14).

In an aspect, method 1600 includes receiving a sense signal from a sensor; and selecting the application duration from a plurality of application durations stored in the data storage location based on the sense signal, as indicated at 1406 (described in connection with FIG. 14).

In an aspect, method 1600 includes receiving the application duration from a user via a user input device; and storing the application duration in the data storage location, as indicated at 1408 (described in connection with FIG. 14).

In an aspect, method 1600 includes generating a stop signal with stop circuitry on the active torso support; and releasing at least a portion of the force applied to the at least a portion of the torso of the subject by the at least one force applying element responsive to the stop signal, as indicated at 1502 (described in connection with FIG. 15). This may further include receiving a stop signal from a user via a user input device; and ending the application time period responsive to receipt of the stop signal, as indicated at 1504 (described in connection with FIG. 15), or receiving a sense signal from a sensor; and ending the application time period based on the sense signal, as indicated at 1506 (described in connection with FIG. 15).

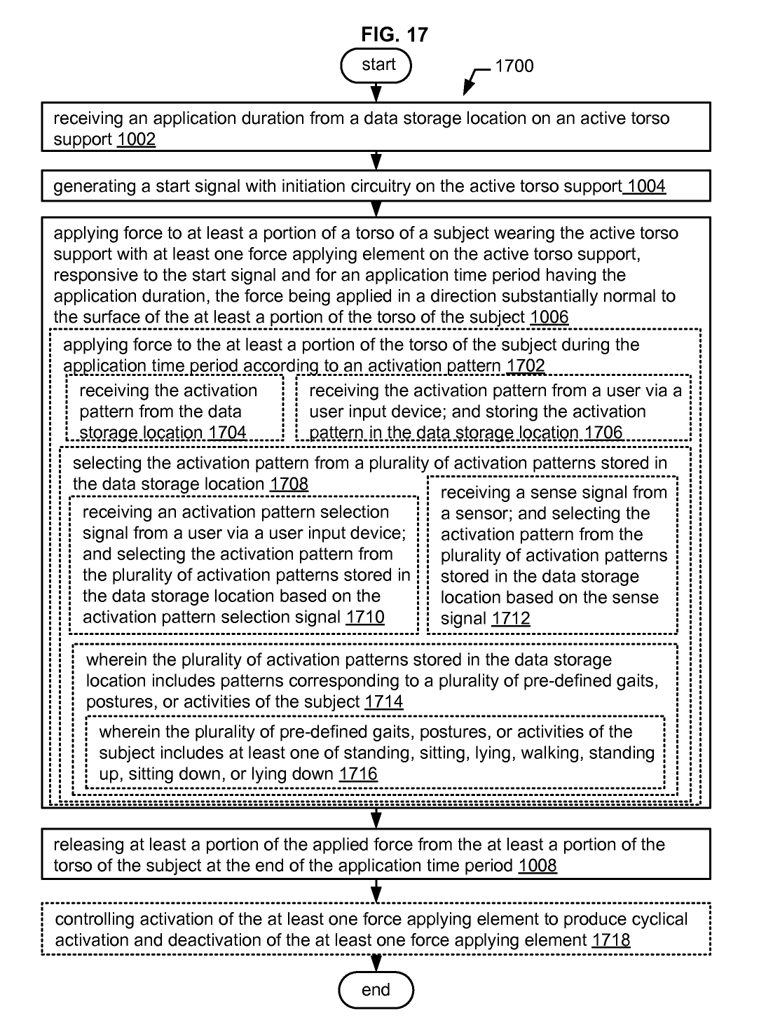

FIG. 17 illustrates a method 1700 of controlling an active torso support, including applying force to the at least a portion of the torso of the subject during the application time period according to an activation pattern, as indicated at 1702. Method 1700 may include receiving the activation pattern from the data storage location, as indicated at 1704, and/or receiving the activation pattern from a user via a user input device; and storing the activation pattern in the data storage location, as indicated at 1706. In an aspect, method 1700 includes selecting the activation pattern from a plurality of activation patterns stored in the data storage location, as indicated at 1708. For example, method 1700 can include receiving an activation pattern selection signal from a user via a user input device; and selecting the activation pattern from the plurality of activation patterns stored in the data storage location based on the activation pattern selection signal, as indicated at 1710, or receiving a sense signal from a sensor; and selecting the activation pattern from the plurality of activation patterns stored in the data storage location based on the sense signal, as indicated at 1712. The plurality of activation patterns stored in the data storage location may include patterns corresponding to a plurality of pre-defined gaits, postures, or activities of the subject, as indicated at 1714. For example, the plurality of pre-defined gaits, postures, or activities of the subject includes at least one of standing, sitting, lying, walking, standing up, sitting down, or lying down, as indicated at 1716.

In a further aspect, a method as described herein (including but not limited to method 1700, can include controlling activation of the at least one force applying element to produce cyclical activation and deactivation of the at least one force applying element, as indicated at 1718.

In various embodiments, methods as described herein may be performed according to instructions implementable in hardware, software, and/or firmware. Such instructions may be stored in non-transitory machine-readable data storage media, for example. Those having skill in the art will recognize that the state of the art has progressed to the point where there is little distinction left between hardware, software, and/or firmware implementations of aspects of systems; the use of hardware, software, and/or firmware is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware in one or more machines, compositions of matter, and articles of manufacture. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In some implementations described herein, logic and similar implementations may include software or other control structures. Electrical circuitry, for example, may have one or more paths of electrical current constructed and arranged to implement various functions as described herein. In some implementations, one or more media may be configured to bear a device-detectable implementation when such media hold or transmit device detectable instructions operable to perform as described herein. In some variants, for example, implementations may include an update or modification of existing software or firmware, or of gate arrays or programmable hardware, such as by performing a reception of or a transmission of one or more instructions in relation to one or more operations described herein. Alternatively or additionally, in some variants, an implementation may include special-purpose hardware, software, firmware components, and/or general-purpose components executing or otherwise invoking special-purpose components.

Implementations may include executing a special-purpose instruction sequence or invoking circuitry for enabling, triggering, coordinating, requesting, or otherwise causing one or more occurrences of virtually any functional operations described herein. In some variants, operational or other logical descriptions herein may be expressed as source code and compiled or otherwise invoked as an executable instruction sequence. In some contexts, for example, implementations may be provided, in whole or in part, by source code, such as C++, or other code sequences. In other implementations, source or other code implementation, using commercially available and/or techniques in the art, may be compiled//implemented/translated/converted into a high-level descriptor language (e.g., initially implementing described technologies in C or C++ programming language and thereafter converting the programming language implementation into a logic-synthesizable language implementation, a hardware description language implementation, a hardware design simulation implementation, and/or other such similar mode(s) of expression). For example, some or all of a logical expression (e.g., computer programming language implementation) may be manifested as a Verilog-type hardware description (e.g., via Hardware Description Language (HDL) and/or Very High Speed Integrated Circuit Hardware Descriptor Language (VHDL)) or other circuitry model which may then be used to create a physical implementation having hardware (e.g., an Application Specific Integrated Circuit). Those skilled in the art will recognize how to obtain, configure, and optimize suitable transmission or computational elements, material supplies, actuators, or other structures in light of these teachings.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In an embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to non-transitory machine-readable data storage media such as a recordable type medium such as a floppy disk, a hard disk drive, a Compact Disc (CD), a Digital Video Disk (DVD), a digital tape, a computer memory, etc. A signal bearing medium may also include transmission type medium such as a digital and/or an analog communication medium (e.g., a fiber optic cable, a waveguide, a wired communications link, a wireless communication link (e.g., transmitter, receiver, transmission logic, reception logic, etc.) and so forth).

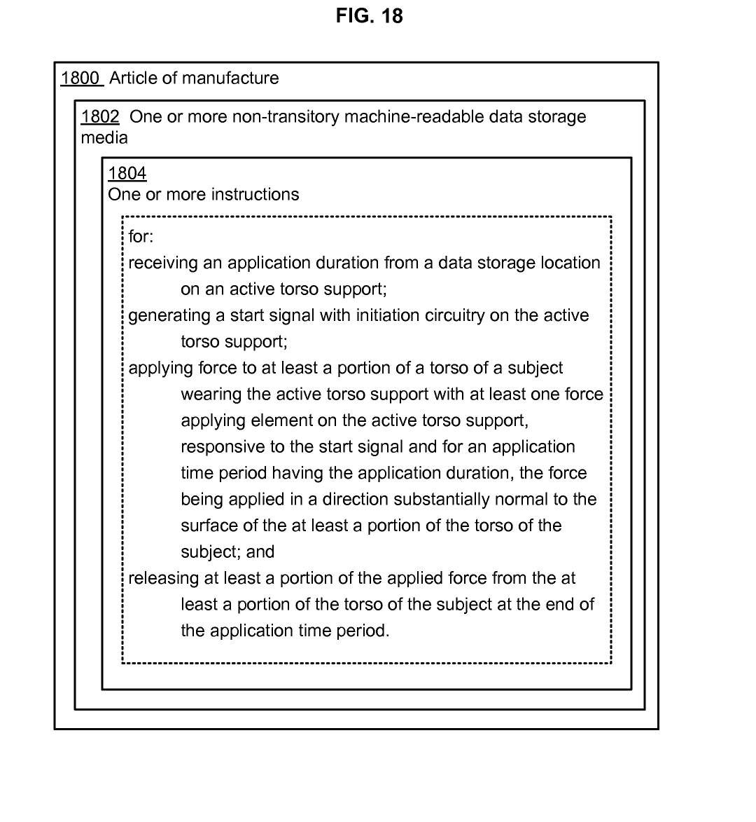

FIG. 18 depicts an article of manufacture 1800 that includes one or more non-transitory machine-readable data storage media 1802 bearing one or more instructions 1804 for: receiving an application duration from a data storage location on an active torso support; generating a start signal with initiation circuitry on the active torso support; controlling at least one force applying element on the active torso support to apply force in a direction substantially normal to the surface of the at least a portion of the torso of the subject, responsive to the start signal and for an application time period having the application duration; and controlling the at least one force applying element to release at least a portion of the applied force from the at least a portion of the torso of the subject at the end of the application time period.

Instructions 1804 depicted in FIG. 18 correspond to the method 1000 shown in FIG. 10. Other variants of methods as depicted in FIGS. 11-17 and as described herein can be implemented through the use of non-transitory machine-readable data storage media bearing one or more suitable instructions.

In an aspect, the one or more non-transitory machine-readable data storage media bear 1802 bear one or more instructions 1804 for performing a method as depicted in FIG. 11, including receiving a signal from a user input device responsive to input from a user, and wherein the one or more instructions for generating a start signal with initiation circuitry on the active torso support include one or more instructions for generating the start signal responsive to receiving the input from the user via the user input device. In an aspect, the one or more non-transitory machine-readable data storage media bear 1802 bear one or more instructions 1804 for performing a method as depicted in FIG. 11, including one or more instructions for receiving a signal indicative of a sensed temperature from a temperature sensor; and modulating the application of force with the at least one force applying element based upon the sensed temperature. In an aspect, the one or more non-transitory machine-readable data storage media bear 1802 bear one or more instructions 1804 for performing a method as depicted in FIG. 11, including one or more instructions for receiving a signal from a timing device; and generating the start signal responsive to receipt of the signal from the timing device. The one or more instructions may include one or more instructions for receiving an interrupt signal, one or more instructions for receiving a time-of-day signal, or one or more instructions for receiving a counter signal. In an aspect, the one or more non-transitory machine-readable data storage media bear 1802 bear one or more instructions 1804 for performing a method as depicted in FIG. 11, including one or more instructions for: generating the start signal responsive to receipt of a power signal from a power source.

In an aspect, the one or more non-transitory machine-readable data storage media bear 1802 bear one or more instructions 1804 for performing a method as depicted in FIG. 12, including one or more instructions for receiving a start time from a data storage location; comparing the time-of-day signal to the start time; and generating the start signal based on the comparison of the time-of-day signal to the start time. In an aspect, the one or more non-transitory machine-readable data storage media bear one or more instructions for: receiving the start time from a user via a user input device; and storing the start time in the data storage location. In an aspect, the one or more non-transitory machine-readable data storage media bear one or more instructions for: receiving a start count from a data storage location; comparing the counter signal to the start count; and generating the start signal based on the comparison of the counter signal to the start count. In addition, the one or more non-transitory machine-readable data storage media may bear one or more instructions for: receiving the start count from a user via a user input device; and storing the start count in the data storage location.

In an aspect, the one or more non-transitory machine-readable data storage media bear 1802 bear one or more instructions 1804 for performing a method as depicted in FIG. 13, including one or more instructions for receiving a signal indicative of at least one gait, posture, or activity of the subject from at least one gait, posture, or activity sensor; and wherein the one or more instructions for generating a start signal with initiation circuitry on the active torso support include one or more instructions for generating the start signal responsive to receiving the signal indicative of the at least one gait, posture, or activity of the subject. In an aspect the one or more instructions for generating the start signal include one or more instructions for: processing the signal from the at least one gait, posture, or activity sensor to detect a change in the gait, posture, or activity of the subject; and generating the start signal responsive to detection of the change in gait, posture, or activity of the subject. The one or more instructions for generating the start signal responsive to detection of the change in gait, posture, or activity of the subject include one or more instructions for generating the start signal in response to detection of a change in posture of the subject from upright to supine, a change in posture of the subject from supine to upright, a change in posture of the subject from sitting to standing, a change in posture of the subject from standing to sitting, a change in activity of the subject, a change in gait of the subject, muscle fatigue in the subject, the subject leaning forward, twisting of the subject's torso, or change in activity level of the subject, e.g., as outlined in connection with FIG. 13.

In an aspect, the one or more non-transitory machine-readable data storage media bear 1802 bear one or more instructions 1804 for performing a method as depicted in FIG. 14, including receiving the application duration from a remote device and storing the application duration in the data storage location; receiving an application duration selection signal from a user via a user input device and selecting the application duration from a plurality of application durations stored in the data storage location based on the application duration selection signal; receiving a sense signal from a sensor and selecting the application duration from a plurality of application durations stored in the data storage location based on the sense signal; or receiving the application duration from a user via a user input device; and storing the application duration in the data storage location.

In an aspect, the one or more non-transitory machine-readable data storage media bear 1802 bear one or more instructions 1804 for performing a method as depicted in FIG. 15, including one or more instructions for generating a stop signal with stop circuitry on the active torso support; and controlling the at least one force applying element to release at least a portion of the force applied to the at least a portion of the torso of the subject responsive to the stop signal. For example, the non-transitory machine-readable data storage media may bear one or more instructions for receiving an input from a user via a user input device and generating the stop signal responsive to receiving the input from the user via the user input device, or one or more instructions for receiving a sense signal from at least one sensor operatively connected to the stop circuitry; and generating the stop signal responsive to receiving the sense signal.

In an aspect, the one or more non-transitory machine-readable data storage media bear 1802 bear one or more instructions 1804 for performing a method as depicted in FIG. 16, including one or more instructions for starting the application time period after a delay time period following generation of the start signal, the delay time period having a delay duration. The one or more non-transitory machine-readable data storage media may bear one or more instructions for receiving the delay duration from the data storage location, receiving the delay duration from a remote device, or receiving the delay duration from a user input device responsive to input of the delay duration by the user. Furthermore, the one or more non-transitory machine-readable data storage media may bear one or more instructions for receiving a delay duration selection signal from a user via a user input device; and selecting the delay duration from a plurality of delay durations stored in the data storage location based on the delay duration selection signal, or receiving a sense signal from a sensor and selecting the delay duration from a plurality of delay durations stored in the data storage location based on the sense signal. In an aspect, the one or more non-transitory machine-readable data storage media bear one or more instructions for receiving the application duration from a remote device; and storing the application duration in the data storage location; receiving an application duration selection signal from a user via a user input device and selecting the application duration from a plurality of application durations stored in the data storage location based on the application duration selection signal; receiving a sense signal from a sensor and selecting the application duration from a plurality of application durations stored in the data storage location based on the sense signal; or receiving the application duration from a user via a user input device and storing the application duration in the data storage location. In an aspect, the one or more non-transitory machine-readable data storage media bear one or more instructions for generating a stop signal with stop circuitry on the active torso support and releasing at least a portion of the force applied to the at least a portion of the torso of the subject by the at least one force applying element responsive to the stop signal. In an aspect, the one or more non-transitory machine-readable data storage media may also bear one or more instructions for receiving an input from a user via a user input device and generating the stop signal responsive to receiving the input from the user via the user input device; or receiving a sense signal from at least one sensor operatively connected to the stop circuitry; and generating the stop signal responsive to receiving the sense signal.

In an aspect, the one or more non-transitory machine-readable data storage media bear 1802 bear one or more instructions 1804 for performing a method as depicted in FIG. 17, including one or more instructions for applying force to the at least a portion of the torso of the subject during the application time period according to an activation pattern. In an aspect, the one or more non-transitory machine-readable data storage media also bear one or more instructions for receiving the activation pattern from the data storage location, and/or receiving the activation pattern from a user via a user input device; and storing the activation pattern in the data storage location. In a further aspect, the one or more non-transitory machine-readable data storage media bear one or more instructions for selecting the activation pattern from a plurality of activation patterns stored in the data storage location, including one or more instructions for receiving an activation pattern selection signal from a user via a user input device and selecting the activation pattern from the plurality of activation patterns stored in the data storage location based on the activation pattern selection signal, or one or more instructions for receiving a sense signal from a sensor and selecting the activation pattern from the plurality of activation patterns stored in the data storage location based on the sense signal. In an aspect, the one or more non-transitory machine-readable data storage media bear one or more instructions for controlling activation of the at least one force applying element to produce cyclical activation and deactivation of the at least one force applying element.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A torso support comprising:
at least one force applying element adapted to apply force to at least a portion of a torso of a subject wearing the torso support;
at least one positioning element adapted to position the at least one force applying element with respect to the torso of the subject such that the at least one force applying element is located between the at least one positioning element and the torso of the subject and arranged to apply force to the at least a portion of the torso of the subject in a direction substantially normal to the surface of the at least a portion of the torso of the subject;
electrical circuitry adapted to control the actuation of the at least one force applying element to apply force to the at least a portion of the torso of the subject, the electrical circuitry including:
initiation circuitry adapted to generate a start signal;
timing circuitry configured to control timing of actuation of the at least one force applying element by the electrical circuitry to:
apply force during an application time period having an application duration responsive to receipt of a start signal; and
release at least a portion of the applied force at the end of the application time period;
a data storage location adapted to store the application duration.

2. The torso support of claim 1, comprising a user input device operatively connected to the initiation circuitry, wherein the initiation circuitry is adapted to generate the start signal responsive to an input from a user on the user input device.

3. The torso support of claim 1, comprising at least one gait, posture, or activity sensor operatively connected to the initiation circuitry, wherein the initiation circuitry is adapted to generate the start signal responsive to a signal indicative of at least one of a gait, posture, or activity of the subject from the at least one gait, posture, or activity sensor.

4-7. (canceled)

8. The torso support of claim 3, wherein the initiation circuitry includes event detection circuitry adapted to process the signal from the at least one gait, posture, or activity sensor to detect a change in the gait, posture or activity of the subject, and wherein the initiation circuitry is adapted to generate the start signal responsive to detection of the change in gait, posture or activity of the subject by the event detection circuitry.

9-18. (canceled)

19. The torso support of claim 1, comprising a timing device operatively connected to the initiation circuitry, wherein the initiation circuitry is adapted to generate the start signal responsive to receipt of a signal from the timing device.

20-24. (canceled)

25. The torso support of claim 1, comprising at least one temperature sensor operatively connected to the electrical circuitry, wherein the electrical circuitry is configured to control the actuation of the at least one force applying element to apply force to the at least a portion of the torso of the subject based at least in part upon the temperature sensed by the temperature sensor.

26-27. (canceled)

28. The torso support of claim 1, comprising a receiver adapted to receive the application duration from a remote device via a wireless connection, wherein the electrical circuitry is adapted to store the application duration received from the remote device in the data storage location.

29-30. (canceled)

31. The torso support of claim 1, comprising at least one sensor operatively connected to the electrical circuitry, wherein the data storage location is adapted to store a plurality of application durations, and wherein the electrical circuitry is adapted to receive a sense signal from the sensor and select the application duration from the plurality of application durations stored in the data storage location based on the sense signal.

32-33. (canceled)

34. The torso support of claim 1, comprising stop circuitry adapted to generate a stop signal, wherein the electrical circuitry is adapted to control the actuation of the at least one force applying element to release at least a portion of the force applied to the at least a portion of the torso of the subject by the at least one force applying element responsive to the stop signal.

35-38. (canceled)

39. The torso support of claim 1, wherein the timing circuitry is configured to control timing of actuation of the at least one force applying element to begin the application time period after a delay time period following receipt of the start signal, the delay time period having a delay duration.

40-46. (canceled)

47. The torso support of claim 1, wherein the electrical circuitry is adapted to control the actuation of the at least one force applying element to apply force to the at least a portion of the torso of the subject during the application time period according to an activation pattern.

48-54. (canceled)

55. The torso support of claim 1, wherein the timing circuitry is configured to control timing of actuation of the at least one force applying element by the electrical circuitry to produce cyclical activation and deactivation of the at least one force applying element.

56. A method of controlling an active torso support comprising:
receiving an application duration from a data storage location on an active torso support;
generating a start signal with initiation circuitry on the active torso support;
applying force to at least a portion of a torso of a subject wearing the active torso support with at least one force applying element on the active torso support, responsive to the start signal and for an application time period having the application duration, the force being applied in a direction substantially normal to the surface of the at least a portion of the torso of the subject; and
releasing at least a portion of the applied force from the at least a portion of the torso of the subject at the end of the application time period.

57-58. (canceled)

59. The method of claim 5856, comprising:
receiving a signal indicative of at least one gait, posture, or activity of the subject from at least one gait, posture, or activity sensor; and
generating the start signal responsive to receiving the signal indicative of the at least one gait, posture, or activity of the subject; wherein generating the start signal includes:
processing the signal from the at least one gait, posture, or activity sensor to detect a change in the gait, posture, or activity of the subject; and
generating the start signal responsive to detection of the change in gait, posture, or activity of the subject.

60-69. (canceled)

70. The method of claim 56, comprising:
receiving a signal indicative of a sensed temperature from a temperature sensor; and
modulating the application of force with the at least one force applying element based upon the sensed temperature.

71. The method of claim 56, comprising:
receiving a signal from a timing device; and
generating the start signal responsive to receipt of the signal from the timing device.

72-78. (canceled)

79. The method of claim 56, comprising:
receiving a power signal from a power source; and
generating the start signal responsive to receipt of the power signal from the power source.

80-83. (canceled)

84. The method of claim 56, comprising:
generating a stop signal with stop circuitry on the active torso support; and
releasing at least a portion of the force applied to the at least a portion of the torso of the subject by the at least one force applying element responsive to the stop signal.

85-86. (canceled)

87. The method of claim 56, comprising starting the application time period after a delay time period following generation of the start signal, the delay time period having a delay duration.

88-101. (canceled)

102. The method of claim 56, comprising applying force to the at least a portion of the torso of the subject during the application time period according to an activation pattern.

103-109. (canceled)

110. The method of claim 56, comprising controlling activation of the at least one force applying element to produce cyclical activation and deactivation of the at least one force applying element.

111. An article of manufacture comprising:
one or more non-transitory machine-readable data storage media bearing one or more instructions for:
receiving an application duration from a data storage location on an active torso support;
generating a start signal with initiation circuitry on the active torso support;
controlling at least one force applying element on the active torso support to apply force in a direction substantially normal to the surface of the at least a portion of the torso of the subject, responsive to the start signal and for an application time period having the application duration; and
controlling the at least one force applying element to release at least a portion of the applied force from the at least a portion of the torso of the subject at the end of the application time period.

112-160. (canceled)

161. The torso support of claim 3, wherein the at least one gait, posture, or activity sensor includes at least one of an accelerometer, an inclinometer, a pressure sensor, and a force sensor.

162. The torso support of claim 8, wherein the event detection circuitry is adapted to process the signal from the at least one gait, posture, or activity sensor to detect at least one of a change in the posture of the subject from upright to supine, a change in the posture of the subject from supine to upright, a change in the posture of the subject from sitting to standing, a change in the posture of the subject from standing to sitting, the subject leaning forward, twisting of the subject's torso, a change in the activity of the subject, muscle fatigue in the subject, a change in the gait of the subject, and a change in an activity level.

163. The torso support of claim 19, wherein the initiation circuitry is configured to generate the start signal based on at least one of receipt of an interrupt signal from the timing device, comparison of a time-of-day signal generated by the timing device with a start time stored in the data storage location, and comparison of a counter signal generated by the timing device to the a count stored in the data storage location.

164. The torso support of claim 1, comprising a power source operatively connected to the initiation circuitry, where the initiation circuitry is adapted to generate the start signal responsive to receipt of a power signal from the power source.

165. The torso support of claim 1, comprising a user input device operatively connected to the electrical circuitry, wherein the electrical circuitry is adapted to receive the application duration from the user via the user input device and store the application duration in the data storage location, or receive an application duration selection signal from the user via the user input device and select the application duration from a plurality of application durations stored in the data storage location based on the application duration selection signal.

166. The torso support of claim 31, wherein the at least one sensor includes at least one of a gait sensor, a posture sensor, an activity sensor, or a temperature sensor.

167. The torso support of claim 34, comprising at least one of:
at least one sensor operatively connected to the stop circuitry, wherein the stop circuitry is adapted to generate the stop signal in response to a sense signal from the sensor; and
a user input device operatively connected to the stop circuitry, wherein the stop circuitry is adapted to generate the stop signal responsive to an input from a user on the user input device.

168. The torso support of claim 39, wherein the data storage location is adapted to store at least one delay duration, and wherein the torso support further comprises at least one of:
a receiver adapted to receive the delay duration from a remote device via a wireless connection, wherein the electrical circuitry is adapted to store the delay duration received from the remote device in the data storage location;
a user input device operatively connected to the electrical circuitry, wherein the electrical circuitry is adapted to receive the delay duration from the user via the user input device and store the delay duration in the data storage location;
a user input device operatively connected to the electrical circuitry, wherein the data storage location is adapted to store a plurality of delay durations, and wherein the electrical circuitry is adapted to receive a delay duration selection signal from the user via the user input device and select the delay duration from the plurality of delay durations stored in the data storage location based on the delay duration selection signal; and
at least one sensor operatively connected to the electrical circuitry, wherein the data storage location is adapted to store a plurality of delay durations, and wherein the electrical circuitry is adapted to receive a sense signal from the sensor and select the delay duration from the plurality of delay durations stored in the data storage location based on the sense signal.

169. The torso support of claim 47, wherein the data storage location is adapted to store at least one activation pattern, and wherein the torso support further comprises:
a user input device operatively connected to the electrical circuitry, wherein the electrical circuitry is adapted to receive the at least one activation pattern from the user via the user input device and store the at least one activation pattern in the data storage location;
a user input device operatively connected to the electrical circuitry, wherein the data storage location is adapted to store a plurality of activation patterns, wherein the electrical circuitry is adapted to receive an activation pattern selection signal from a user via the user input device, select the activation pattern from the plurality of activation patterns based on the activation pattern selection signal, and control the actuation of the at least one force applying element to apply force to the at least a portion of the torso of the subject during the application time period according to an activation pattern selected from the plurality of activation patterns; and
at least one sensor operatively connected to the electrical circuitry, wherein the data storage location is adapted to store a plurality of activation patterns, and wherein the electrical circuitry is adapted to receive a sense signal from the at least one sensor and select the activation pattern from the plurality of activation patterns based on the sense signal.

170. The method of claim 56, comprising at least one of:
receiving an input from a user via a user input device and generating the start signal responsive to receiving the input from the user via the user input device; and
receiving a signal indicative of at least one gait, posture, or activity of the subject from at least one gait, posture, or activity sensor and generating the start signal responsive to receiving the signal indicative of the at least one gait, posture, or activity of the subject.

171. The method of claim 59, wherein generating the start signal includes generating the start signal in response to detection of at least one of a change in posture of the subject from upright to supine, a change in posture of the subject from supine to upright, a change in posture of the subject from sitting to standing, a change in posture of the subject from standing to sitting, a change in activity of the subject, a change in gait of the subject, muscle fatigue in the subject, subject leaning forward, twisting of the subject's torso, and change in activity level of the subject.

172. The method of claim 71, wherein receiving the signal from the timing device includes at least one of a receiving an interrupt signal, receiving a time-of-day signal and receiving a counter signal.

173. The method of claim 56, comprising at least one of:
receiving the application duration from a remote device and storing the application duration in the data storage location;
receiving an application duration selection signal from a user via a user input device and selecting the application duration from a plurality of application durations stored in the data storage location based on the application duration selection signal;
receiving a sense signal from a sensor and selecting the application duration from a plurality of application durations stored in the data storage location based on the sense signal; and
receiving the application duration from a user via a user input device and storing the application duration in the data storage location.

174. The method of claim 84, comprising at least one of:
receiving an input from a user via a user input device and generating the stop signal responsive to receiving the input from the user via the user input device; and
receiving a sense signal from at least one sensor operatively connected to the stop circuitry and generating the stop signal responsive to receiving the sense signal.

175. The method of claim 87, comprising at least one of:
receiving the delay duration from the data storage location;
receiving the delay duration from a remote device;
receiving a delay duration selection signal from a user via a user input device and selecting the delay duration from a plurality of delay durations stored in the data storage location based on the delay duration selection signal;
receiving a sense signal from a sensor and selecting the delay duration from a plurality of delay durations stored in the data storage location based on the sense signal; and
receiving the delay duration from a user input device responsive to input of the delay duration by the user.

176. The method of claim 87, comprising at least one of:
receiving the application duration from a remote device and storing the application duration in the data storage location;
receiving an application duration selection signal from a user via a user input device and selecting the application duration from a plurality of application durations stored in the data storage location based on the application duration selection signal;
receiving a sense signal from a sensor and selecting the application duration from a plurality of application durations stored in the data storage location based on the sense signal;
receiving the application duration from a user via a user input device and storing the application duration in the data storage location;
receiving a sense signal from a sensor and ending the application time period based on the sense signal; and
receiving a stop signal from a user via a user input device and ending the application time period responsive to receipt of the stop signal.

177. The method of claim 87, comprising at least one of:
generating a stop signal with stop circuitry on the active torso support and releasing at least a portion of the force applied to the at least a portion of the torso of the subject by the at least one force applying element responsive to the stop signal;
receiving an input from a user via a user input device and generating the stop signal responsive to receiving the input from the user via the user input device; and
receiving a sense signal from at least one sensor operatively connected to the stop circuitry and generating the stop signal responsive to receiving the sense signal.

178. The method of claim 102, comprising at least one of:
receiving the activation pattern from the data storage location;
receiving the activation pattern from a user via a user input device and storing the activation pattern in the data storage location; and
selecting the activation pattern from a plurality of activation patterns stored in the data storage location.

179. The method of claim 178, comprising at least one of:
receiving an activation pattern selection signal from a user via a user input device and selecting the activation pattern from the plurality of activation patterns stored in the data storage location based on the activation pattern selection signal; and
receiving a sense signal from a sensor and selecting the activation pattern from the plurality of activation patterns stored in the data storage location based on the sense signal.
